(19) United States
(12) Patent Application Publication
(10) Pub. No.: US 2009/0246205 A1
(43) Pub. Date: Oct. 1, 2009

(54) Inhibition of macrophage-stimulating protein receptor (ron)

(75) Inventors: Daniel Pereira, Toronto (CA); Dan Lu, Plainsboro, NJ (US)

Correspondence Address: ELI LILLY & COMPANY, PATENT DIVISION, P.O. BOX 6288, INDIANAPOLIS, IN, 46206-6288, US

(73) Assignee: ImClone Systems, Inc, New York, NY (US)

(21) Appl. No.: 11/596,030

(22) PCT Filed: May 13, 2005
(86) PCT No.: PCT/US05/16920

§ 371 Date: Sep. 10, 2007

Related U.S. Application Data
(60) Provisional application No. 60/571,648, filed on May 13, 2004.

(51) Int. Cl. A61K 39/395; C07K 16/18; C07H 21/00; C12N 15/63; C12N 5/00; C12P 21/00; G01N 33/53; A61P 35/00; A61P 29/00

(52) U.S. Cl. 424/142.1; 530/388.22; 536/22.1; 435/320.1; 435/325; 435/69.6; 424/144.1; 436/501; 424/178.1; 530/389.2

(57) ABSTRACT

The present invention relates to methods for treatment of tumors and other diseases in a mammal comprising administration of antibodies specific for Macrophage-Stimulating Protein Receptor (“MSP-R” or “RON”). The present invention further provides for compositions comprising antibodies or antibody fragments specific for RON, including human antibodies, that inhibit RON activation.

### FIELD OF THE INVENTION

The present invention relates to methods for treatment of tumors and other diseases in a mammal comprising administration of antibodies specific for Macrophage-Stimulating Protein Receptor (“MSP-R” or “RON”). The present invention further provides for compositions comprising antibodies or antibody fragments specific for RON, including human antibodies, that inhibit RON activation.

### BACKGROUND OF THE INVENTION

RON belongs to the c-met family of receptor tyrosine kinases. RON is a heterodimeric protein comprised of an extracellular alpha chain and a transmembrane beta chain. RON is first expressed as a single chain precursor, followed by cleavage into the alpha and the beta chains (1). It is believed that the beta chain is required for binding of MSP to the receptor, and Kringle domains of 2 and 3 are required for RON/MSP interaction. U.S. Publ. No. 2003/0073656. The extracellular domain of RON is thought to have little homology with the corresponding domains of the c-met family receptors. Indeed, binding of Hepatocyte Growth Factor (HGF), which stimulates other receptors in the c-met family, to the RON receptor, does not stimulate tyrosine kinase activity. WO02/083047.

RON is thought to have a role in cell migration, shape change and invasion (1). An earlier publication however found limited role for RON for inducing transformation, but found promotion of invasive growth by RON activation (16). U.S. Publ. No. 2003/0073656 speculates that activation of RON may play a role in diseases to the liver, biliary tract, bile ducts, gall bladder and related hepatobiliary system.

Mutations, deletions, gene rearrangements and alternative mRNA splicing may cause activation of RON without any ligand binding (1). Variations in the tyrosine kinase domain of RON may play an important role in activation of RON (1). Cloning of RON from various cancer cell lines, has shown RON activation due to various defects in the mRNA encoding for RON.

In addition to c-met's ligand (HGF), the ligand for RON (Macrophage-Stimulating Protein; MSP aka. HGF-like protein) is a member of the kringle-domain plasminogen-related protein family (1). As its name implies, MSP was originally found to stimulate macrophages by a variety of means (2,3). For example, addition of MSP to certain RON-expressing macrophages induced shape changes, chemotaxis, macropinocytosis, phagocytosis and immune mediator production (4, 5, 6). RON was also found to be expressed in epithelial cells such as keratinocytes where MSP was shown to phosphorylate RON and activate a number of signaling pathways that elicited cell adhesion/motility, anti-apoptotic and proliferative responses (7,8). Within the last few years, over-expression of RON has been observed in several epithelial tumors and cell lines (ex. colon (9, 10, 11), lung (12), breast (13)). In a recent study, lung tumors developed in transgenic mice engineered to over-express RON in their lungs (14,15).

Studies to address whether inhibition of RON could abrogate tumor or cancer cell line growth have not been reported.

### SUMMARY OF THE INVENTION

The present invention relates to methods for treatment of tumors and other diseases in a mammal comprising administration of antibodies specific for Macrophage-Stimulating Protein Receptor (“MSP-R” or “RON”). The present invention further provides for compositions comprising antibodies or antibody fragments specific for RON, including human antibodies, that inhibit RON activation.

The present invention further provides a monoclonal antibody, or fragment thereof, specific for RON comprising one or more heavy chain CDR sequences selected from the group consisting of SEQ ID NO: 2 (SYAMH) for CDR1; SEQ ID NO: 4 (VISYDGSNKYYADSVKG) for CDR2 and SEQ ID NO: 6 for CDR3 (FSGWPNNYYYYGMDV).

The present invention further provides a monoclonal antibody, or a fragment thereof, specific for RON comprising one or more light chain CDR sequences selected from the group consisting of SEQ ID NO: 11 for CDR1 (RSSQSLLHSNGFNYVD); SEQ ID NO: for CDR2 (FGSYRAS) and SEQ ID NO: 15 for CDR3 (MQALQTPPWT).

The present invention further provides a monoclonal antibody, or a fragment thereof, specific for RON comprising one or more light chain CDR sequences selected from the group consisting of SEQ ID NO: 50 for CDR1 (RSSQSLLHSNGYNYLD); SEQ ID NO: 52 for CDR2 (LGSNRAS) and SEQ ID NO: for CDR3 (MQALQTPRT).

The present invention further provides a monoclonal antibody, or fragment thereof, specific for RON comprising one or more heavy chain CDR sequences selected from the group consisting of SEQ ID NO: 20 (SHYWS) for CDR1; SEQ ID NO: 23 (YIYYSGSTNYNPSLKS) for CDR2 and SEQ ID NO: for CDR3 (IPNYYDRSGYYPGYWYFDL).

The present invention further provides a monoclonal antibody, or fragment thereof, specific for RON comprising one or more light chain CDR sequences selected from the group consisting of SEQ ID NO: for CDR1 (TLRSGFNVDSYRIS); SEQ ID NO: for CDR2 (YKSDSDK) and SEQ ID NO: 18 for CDR3 (MIWHSSAWV).

The present invention further provides isolated nucleic acids encoding RON specific antibodies and antibody fragments. Also provided are expression vectors, host cells comprising the expression vectors and methods for producing RON specific antibodies comprising culturing a host cell.

The present invention further provides a pharmaceutical compositions comprising RON specific monoclonal antibodies, or fragment thereof. Such compositions may be used in methods for inhibiting growth of mammalian tumor cells that express RON comprising administering an effective amount of an antibody or a fragment thereof specific for RON. The present invention further provides a method for inhibiting metastatic activity of mammalian tumor cells that express RON, comprising administering an effective amount of an antibody or a fragment thereof specific for RON. The present invention provides a method for treating inflammation mediated by RON activity in a mammal comprising administering to the mammal an antibody or an antibody fragment specific for RON.

In addition to administration of RON specific antibodies, the methods of the present invention further provide for administering a small organic molecule, wherein the small organic molecule is a chemotherapeutic agent, anti-angiogenesis agent or inhibits activation of RON.

In addition to administration of RON specific antibodies, the methods of the present invention further provide for administering one or more antibodies specific to a receptor tyrosine kinase, such as EGFR or VEGFR.

The present invention provides a therapeutic composition for inhibition of growth of tumor cells that express RON in a mammal comprising an antibody, or fragment thereof, specific for RON.

The present invention further provides a method for detecting presence of RON comprising contacting RON with the above antibody pr a fragment thereof.

### BRIEF DESCRIPTION OF THE FIGURES

FIG. 1 provides a chart plotting size of tumors against time in mice after administration of IMC-41A10.

FIG. 2 defines various SEQ ID Nos. including those of the antibodies of the present invention.

FIG. 3 is a western blot illustrating inhibition of MSP induced phosphorylation by IMC-41A10.

### DETAILED DESCRIPTION OF THE INVENTION

The present invention provides a method of inhibiting growth, proliferation, metastatic activity (i.e. migration and/or invasion) of tumor cells that express RON by administration of an effective amount of an antibody or a fragment thereof that inhibits activation of RON. The invention also provides therapeutic compositions of an antibody, or fragment thereof, specific for RON. Further, the present invention provides fully human antibodies to the human RON receptor tyrosine kinase. Such antibodies include but are not limited to IMC-41A2, IMC-41A10 and IMC-41B12, and fragments thereof.

Naturally occurring antibodies typically have two identical heavy chains and two identical light chains, with each light chain covalently linked to a heavy chain by an inter-chain disulfide bond and multiple disulfide bonds further link the two heavy chains to one another. Individual chains can fold into domains having similar sizes (110-125 amino acids) and structures, but different functions. The light chain can comprise one variable domain (V_{L}) and/or one constant domain (C_{L}). The heavy chain can also comprise one variable domain (V_{H}) and/or, depending on the class or isotype of antibody, three or four constant domains (C_{H}1, C_{H}2, C_{H}3 and C_{H}4). In humans, the isotypes are IgA, IgD, IgE, IgG, and IgM, with IgA and IgG further subdivided into subclasses or subtypes (IgA₁₋₂ and IgG₁₋₄).

Generally, the variable domains show considerable amino acid sequence variability from one antibody to the next, particularly at the location of the antigen-binding site. Three regions, called hypervariable or complementarity-determining regions (CDRs), are found in each of V_{L} and V_{H}, which are supported by less variable regions called framework variable regions.

The portion of an antibody consisting of V_{L} and V_{H} domains is designated Fv (Fragment variable) and constitutes the antigen-binding site. Single chain Fv (scFv) is an antibody fragment containing a V_{L} domain and a V_{H} domain on one polypeptide chain, wherein the N terminus of one domain and the C terminus of the other domain are joined by a flexible linker (see, e.g., U.S. Pat. No. 4,946,778 (Ladner et al.); WO 88/09344, (Huston et al.). WO 92/01047 (McCafferty et al.) describes the display of scFv fragments on the surface of soluble recombinant genetic display packages, such as bacteriophage.

The peptide linkers used to produce the single chain antibodies can be flexible peptides selected to assure that the proper three-dimensional folding of the V_{L} and V_{H} domains occurs. The linker is generally 10 to 50 amino acid residues. Preferably, the linker is 10 to 30 amino acid residues. More preferably the linker is 12 to 30 amino acid residues. Most preferably is a linker of 15 to 25 amino acid residues. An example of such linker peptides includes repeats of four Glycines followed by Serine.

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one V_{H} and one V_{L} domain covalently linked by a first peptide linker, can be covalently linked by at least one or more peptide linker to form a multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of amino acid residues is about one hundred.

Two single chain antibodies can be combined to form a diabody, also known as a bivalent dimer. Diabodies have two chains and two binding sites, and can be monospecific or bispecific. Each chain of the diabody includes a V_{H} domain connected to a V_{L} domain. The domains are connected with linkers that are short enough to prevent pairing between domains on the same chain, thus driving the pairing between complementary domains on different chains to recreate the two antigen-binding sites.

Three single chain antibodies can be combined to form triabodies, also known as trivalent trimers. Triabodies are constructed with the amino acid terminus of a V_{L} or V_{H} domain directly fused to the carboxyl terminus of a V_{L} or V_{H} domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

Fab (Fragment, antigen binding) refers to the fragments of the antibody consisting of V_{L} C_{L} V_{H} C_{H1} domains. Those generated following papain digestion simply are referred to as Fab and do not retain the heavy chain hinge region. Following pepsin digestion, various Fabs retaining the heavy chain hinge are generated. Those fragments with the interchain disulfide bonds intact are referred to as F(ab′)₂, while a single Fab′ results when the disulfide bonds are not retained. F(ab′)₂ fragments have higher avidity for antigen that the monovalent Fab fragments.

Fc (Fragment crystallization) is the designation for the portion or fragment of an antibody that comprises paired heavy chain constant domains. In an IgG antibody, for example, the Fc comprises C_{H2} and C_{H3} domains. The Fc of an IgA or an IgM antibody further comprises a C_{H4} domain. The Fc is associated with Fc receptor binding, activation of complement-mediated cytotoxicity and antibody-dependent cellular-cytotoxicity (ADCC). For antibodies such as IgA and IgM, which are complexes of multiple IgG like proteins, complex formation requires Fc constant domains.

Finally, the hinge region separates the Fab and Fc portions of the antibody, providing for mobility of Fabs relative to each other and relative to Fc, as well as including multiple disulfide bonds for covalent linkage of the two heavy chains.

Thus, antibodies specific to RON include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabodies, triabodies, and the like that bind specifically with antigens.

Each domain of the antibodies of this invention can be a complete antibody with the heavy or light chain variable domain, or it can be functionally the same or a mutant or derivative of a naturally-occurring domain, or a synthetic domain constructed, for example, in vitro using a technique such as one described in WO 93/11236 (Griffiths et al.). For instance, it is possible to join together domains corresponding to antibody variable domains, which are missing at least one amino acid. The important characterizing feature is the ability of each domain to associate with a complementary domain to form an antigen-binding site. Accordingly, the terms variable heavy and light chain fragment should not be construed to exclude variants that do not have a material effect on specificity.

As used herein, “antibodies” and “antibody fragments” includes modifications that retain specificity for the RON receptor. Such modifications include, but are not limited to, conjugation to an effector molecule such as a chemotherapeutic agent (e.g., cisplatin, taxol, doxorubicin) or cytotoxin (e.g., a protein, or a non-protein organic chemotherapeutic agent). The antibodies can be modified by conjugation to detectable reporter moieties. Also included are antibodies with alterations that affect non-binding characteristics such as half-life (e.g., pegylation).

Proteins and non-protein agents may be conjugated to the antibodies by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990) for the conjugation of doxorubicin and those described by Amon et al., Adv. Exp. Med. Biol. 303, 79-90 (1991) and by Kiseleva et al., Mol. Biol. (USSR) 25, 508-514 (1991) for the conjugation of platinum compounds.

Antibody specificity refers to selective recognition of the antibody for a particular epitope of an antigen. Antibodies, or fragments thereof, of the present invention, for example, can be monospecific or bispecific. Bispecific antibodies (BsAbs) are antibodies that have two different antigen-binding specificities or sites. Where an antibody has more than one specificity, the recognized epitopes can be associated with a single antigen or with more than one antigen. Thus, the present invention provides bispecific antibodies, or fragments thereof, that bind to two different antigens, with at least one specificity for RON.

Specificity of antibodies, or fragments thereof, for RON can be determined based on affinity and/or avidity. Affinity, represented by the equilibrium constant for the dissociation of an antigen with an antibody (K_{d}), measures the binding strength between an antigenic determinant and an antibody-binding site. Avidity is the measure of the strength of binding between an antibody with its antigen. Avidity is related to both the affinity between an epitope with its antigen binding site on the antibody, and the valence of the antibody, which refers to the number of antigen binding sites of a particular epitope. Antibodies typically bind with a dissociation constant (K_{d}) of about 10⁻⁵ to about 10⁻¹¹ liters/mol (e.g., K_{D}<100 nM). Any K_{d} less than about 10⁴ liters/mol is generally considered to indicate nonspecific binding. The lesser the value of the K_{d}, the stronger the binding strength between an antigenic determinant and the antibody binding site.

RON may be isolated from various sources to raise an immune response, such as from cells that express RON: colon, pancreatic, prostate, stomach, lung, liver, ovarian, kidney, breast and brain, and in general epithelial and neuroendocrine. Also, a synthetic receptor peptide may be obtained using commercially available machines and the corresponding amino acid sequence. A further alternative still, is that DNA encoding a RON such as a cDNA or a fragment thereof, may be cloned and expressed and the resulting polypeptide recovered and used as an immunogen to raise an antibody of the invention. In order to prepare RON against which the antibodies are made, nucleic acid molecules that encode RON, or portions thereof, especially the extracellular portions thereof (particularly alpha and beta portion), may be inserted into known vectors for expression in host cells using standard recombinant DNA techniques. Similarly, antibodies against ligands of RON, particularly MSP, may be prepared.

The sequences for RON and its ligand MSP are publicly available and can readily be used for antibody preparation. Antibodies may also be produced against variants/mutants of RON or MSP. Of interest are antibodies to epitopes present on extracellular domains of variants and mutants. An altered RON receptor differing by an in-frame deletion of 109 amino acids in the extracellular domain has been shown to be constitutively activated (1). Antibodies may for example be generated against such altered RON receptor.

Antibodies specific to RON may be prepared by immunizing a mammal with RON. The soluble receptors may be used by themselves as immunogens, or attached to a carrier protein or other objects, such as beads, i.e. sepharose beads. After the mammal has produced antibodies, a mixture of antibody producing cells, such as splenocytes, are isolated. Monoclonal antibodies may be produced by isolating individual antibody-producing cells from the mixture and immortalizing them by, for example, fusing them with tumor cells, such as myeloma cells. The resulting hybridomas are preserved in culture, and express monoclonal antibodies, which are harvested from the culture medium.

Further, antibodies and antibody fragments of the invention can be obtained by standard hybridoma technology (Harlow & Lane, ed., Antibodies: A Laboratory Manual, Cold Spring Harbor, 211-213 (1998), which is incorporated by reference herein) using transgenic mice (e.g., KM mice from Medarex, San Jose, Calif.) that produce human immunoglobulin heavy and light chains. In a preferred embodiment, a substantial portion of the human antibody producing genome is inserted into the genome of the mouse, and is rendered deficient in the production of endogenous murine antibodies. Such mice may be immunized subcutaneously (s.c.) with RON in complete Freund's adjuvant. The antibodies of this invention can be fused to additional amino acid residues. Such amino acid residues can be a peptide tag, perhaps to facilitate isolation. Other amino acid residues for homing of the antibodies to specific organs or tissues are also contemplated.

Anti-RON antibodies according to the present invention can be isolated from a phage display library such as one constructed from human heavy chain and light chain variable region genes. For example, a variable domain of the invention can be obtained from peripheral blood lymphocytes that contains a rearranged variable region gene. Alternatively, variable domain portions, such as CDR and FW regions, can be obtained from different human sequences.

The antibodies specific to RON bind to RON with a K_{d} of preferably about 1×10⁻⁹ M⁻¹ or less, more preferably about 1×10⁻¹⁰ M⁻¹ or less, and most preferably about 1×10⁻¹¹ M⁻¹ or less.

Antibodies, or fragments thereof, specific for RON, inhibit activation of the receptor. Inhibiting a receptor means preventing the activation of the intrinsic kinase activity of the receptor to transduce a signal. A reliable assay for RON is the inhibition of receptor phosphorylation.

The present invention is not limited by any particular mechanism of RON inhibition. Such inhibition for example may occur by an antibody blocking access to certain epitopes by a ligand, or by changing conformation of RON in a manner that the ligand, particularly MSP, can not activate the receptor even though it can bind to the receptor. U.S. Pat. No. 6,165,464 lists various possible mechanisms for such inhibition, including binding to the ligand itself, down regulating the receptor, inhibiting the tyrosine kinase activity of the receptor, or illiciting a cytotoxic response. Down regulation may occur when cells that express RON, particularly those that overexpress (including differentially express) RON, decrease the number of RON receptor tyrosine kinases on their surface. Matrix metalloproteinases, which function in tumor cell invasion and metastasis, may also be down regulated by the antibodies of the present invention.

RON inhibition has various effects, including inhibition, diminution, inactivation and/or disruption of growth (proliferation and differentiation), angiogenesis (blood vessel recruitment, invasion, and metastasis), and cell motility and metastasis (cell adhesion and invasiveness).

The invention also contemplates antibodies that bind to and inactivate variant or mutated RON receptor tyrosine kinases that are active without ligand binding. A mammal suffering from a RON related disease may for example express both wild type and variant RON, with a disproportionate amount of the variant receptor. Of interest are sequences of variants/mutants differing in the extracellular domain, such as those having deletions within the extracellular domain, as disclosed by Wang (1) (9). Thus RON inhibition may involve wild type and/or variant RON (point mutations, deletions, alternative splicing, etc.).

RON activation may occur through dimerization and activation with other RTKs such as c-met or EGFR. Thus, RON inhibition may also include inhibition of heterodimerization between RON and other RTKs such as EGFR or c-met. Such inhibition may also include inhibition of signaling by a formed heterodimer of RON and EGF or c-met as an example. Such dimerization may have been induced in a ligand dependent fashion, such as by MSP, HGF or EGF binding to their receptors and inducing dimerization.

One measure of RON inhibition is inhibition of the tyrosine kinase activity of the receptor. Tyrosine kinase inhibition can be determined using well-known methods; for example, by measuring the autophosphorylation level of recombinant kinase receptor, and/or phosphorylation of natural or synthetic substrates. Thus, phosphorylation assays are useful in determining inhibiting antibodies in the context of the present invention. Phosphorylation can be detected, for example, using an antibody specific for phosphotyrosine in an ELISA assay or on a western blot. Some assays for tyrosine kinase activity are described in Panek et al., J. Pharmacol. Exp. Thera. 283: 1433-44 (1997) and Batley et al., Life Sci. 62:143-50 (1998).

In addition, methods for detection of protein expression can be utilized to determine RON inhibition. These methods include immunohistochemistry (IHC) for detection of protein expression, fluorescence in situ hybridization (FISH) for detection of gene amplification, competitive radioligand binding assays, solid matrix blotting techniques, such as Northern and Southern blots, reverse transcriptase polymerase chain reaction (RT-PCR) and ELISA.

Another measure of RON inhibition of phosphorylation of downstream substrates of RON. Accordingly, the level of phosphorylation of MAPK or Akt can be measured.

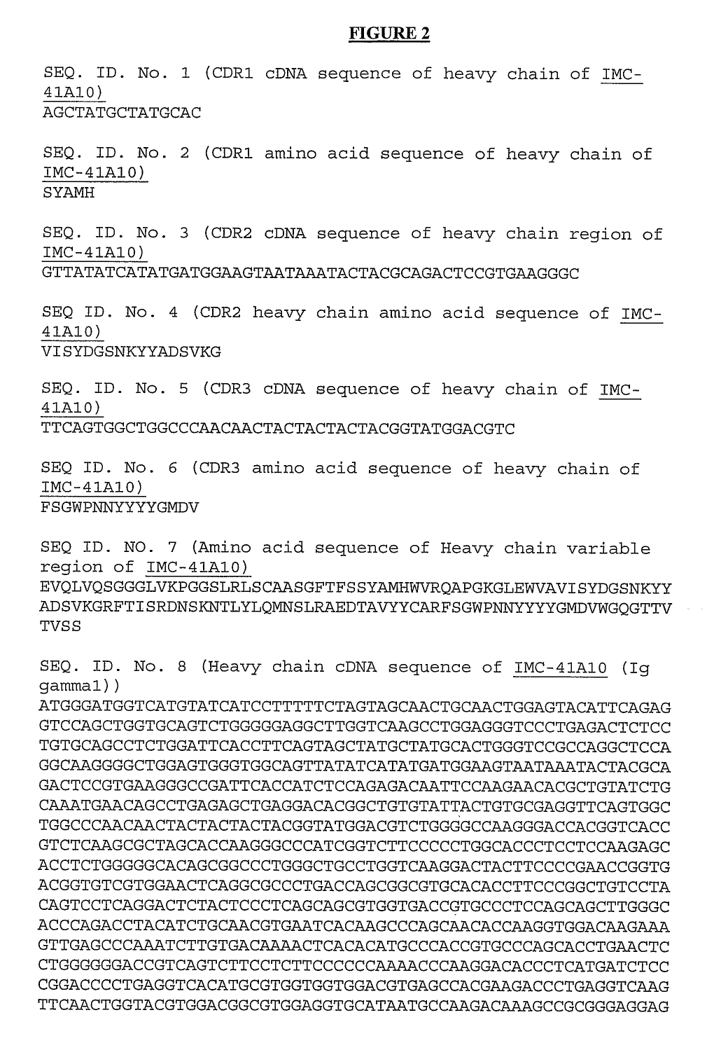

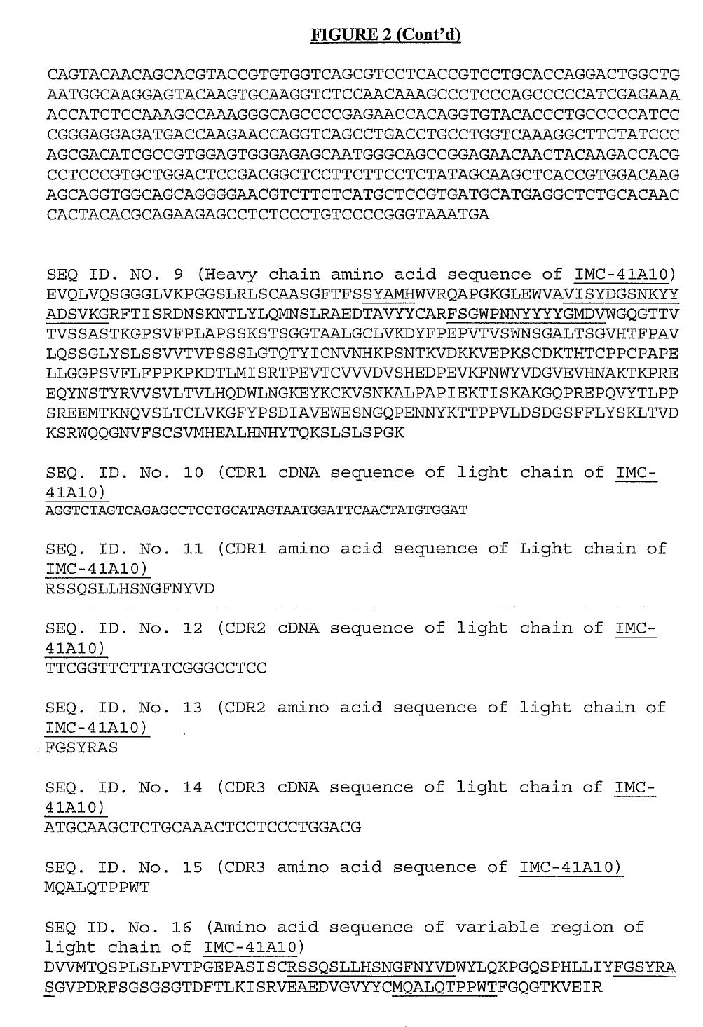

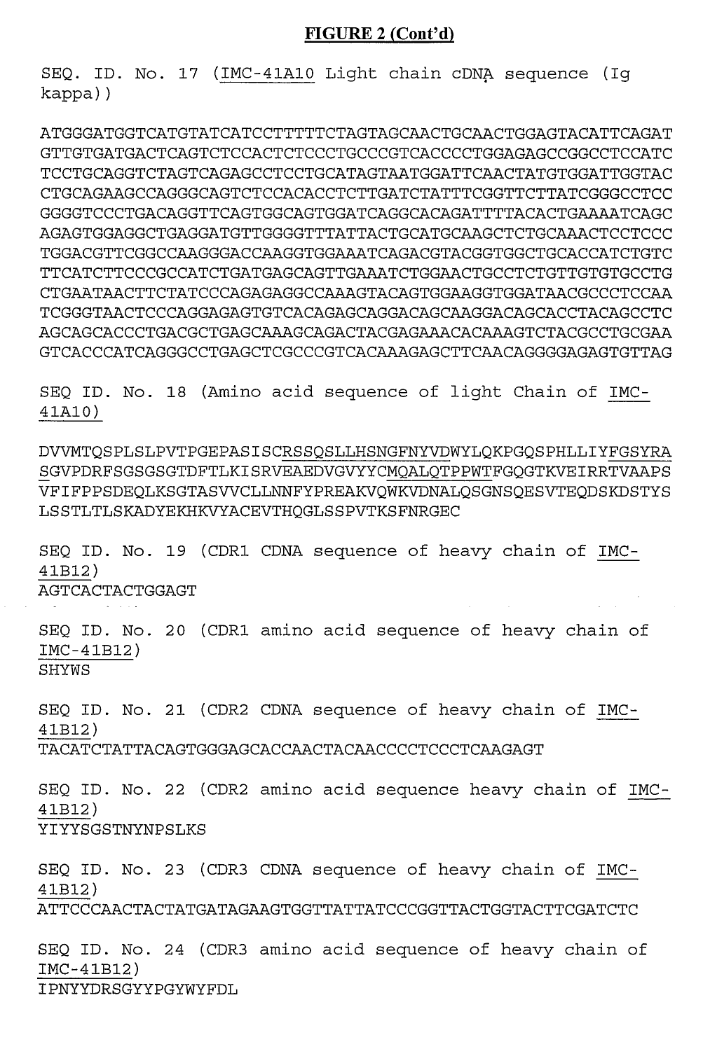

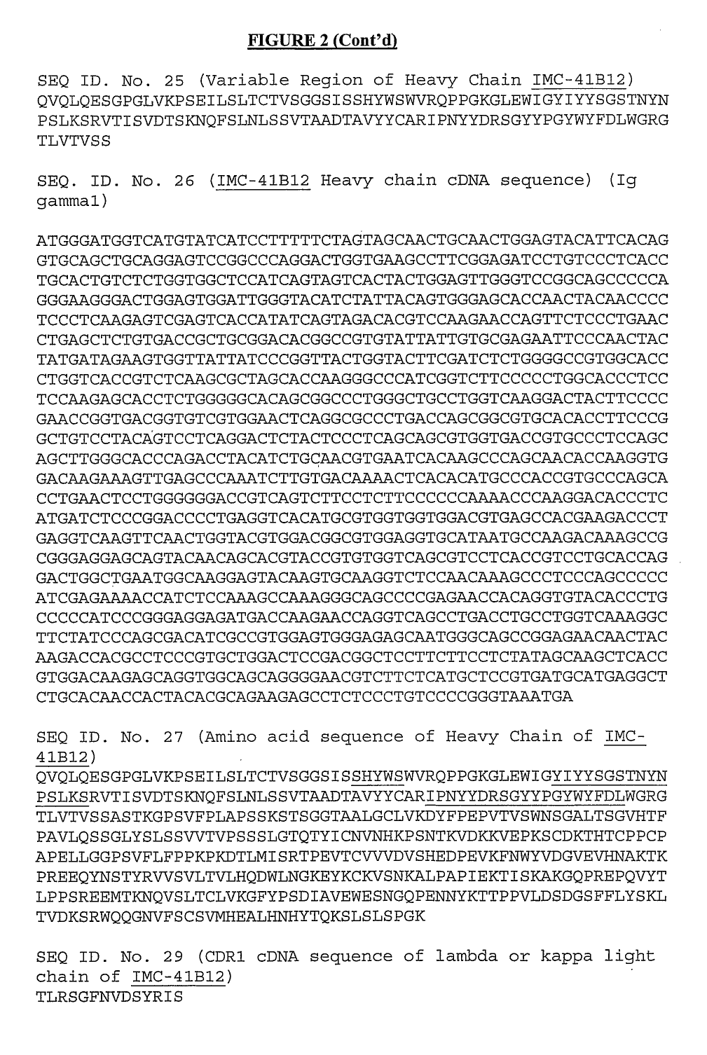

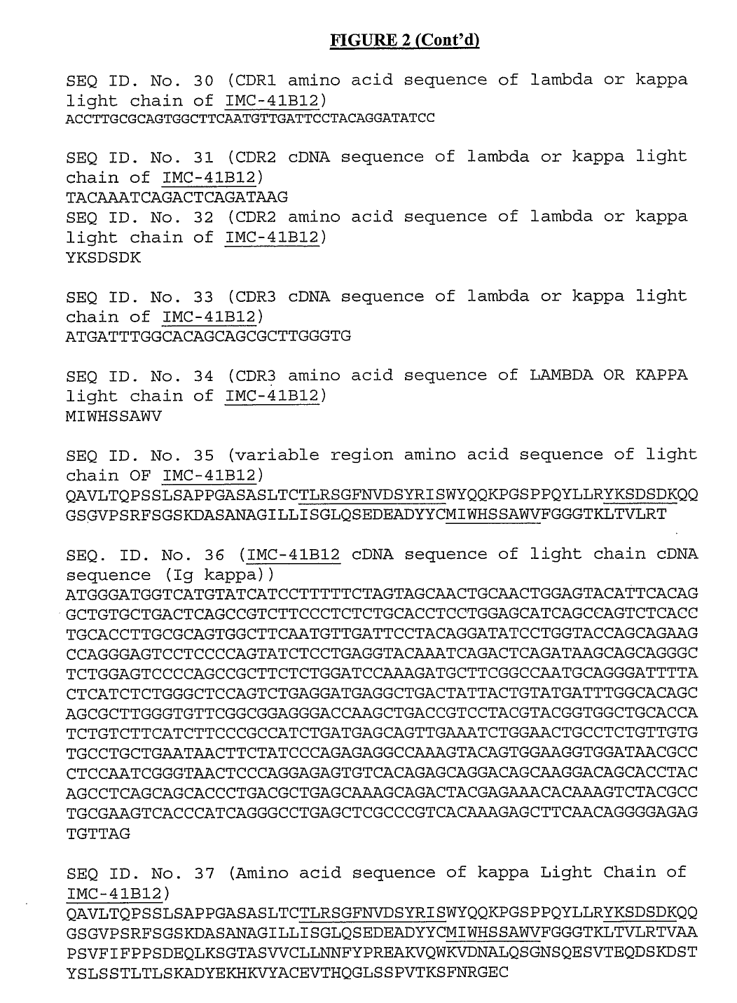

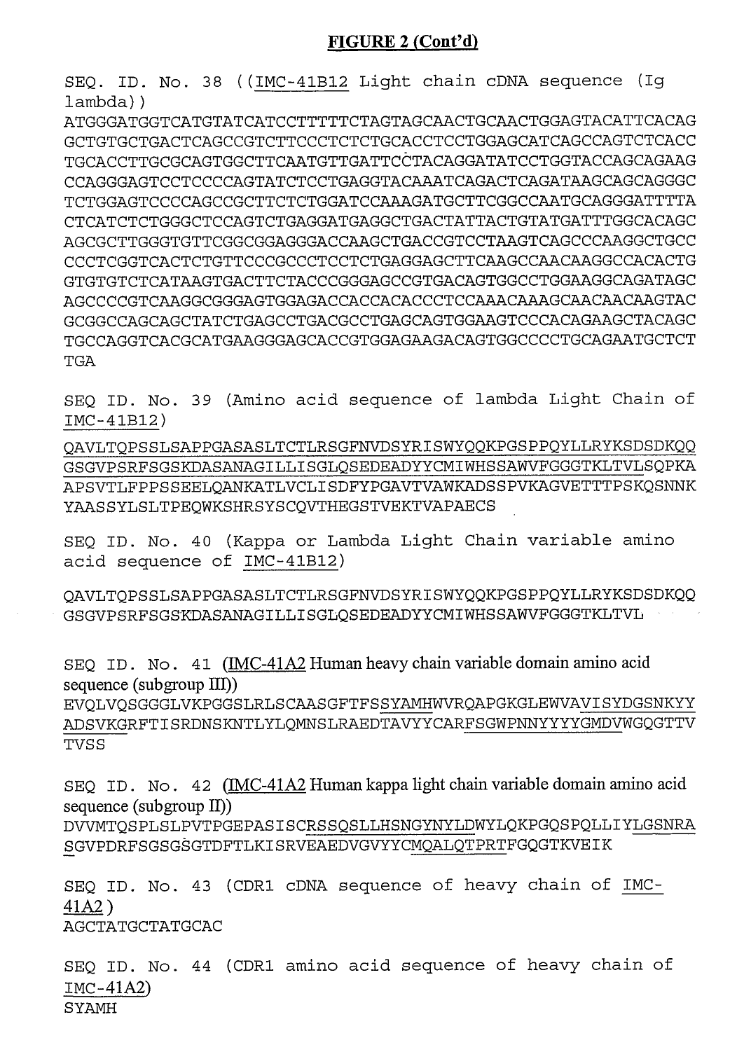

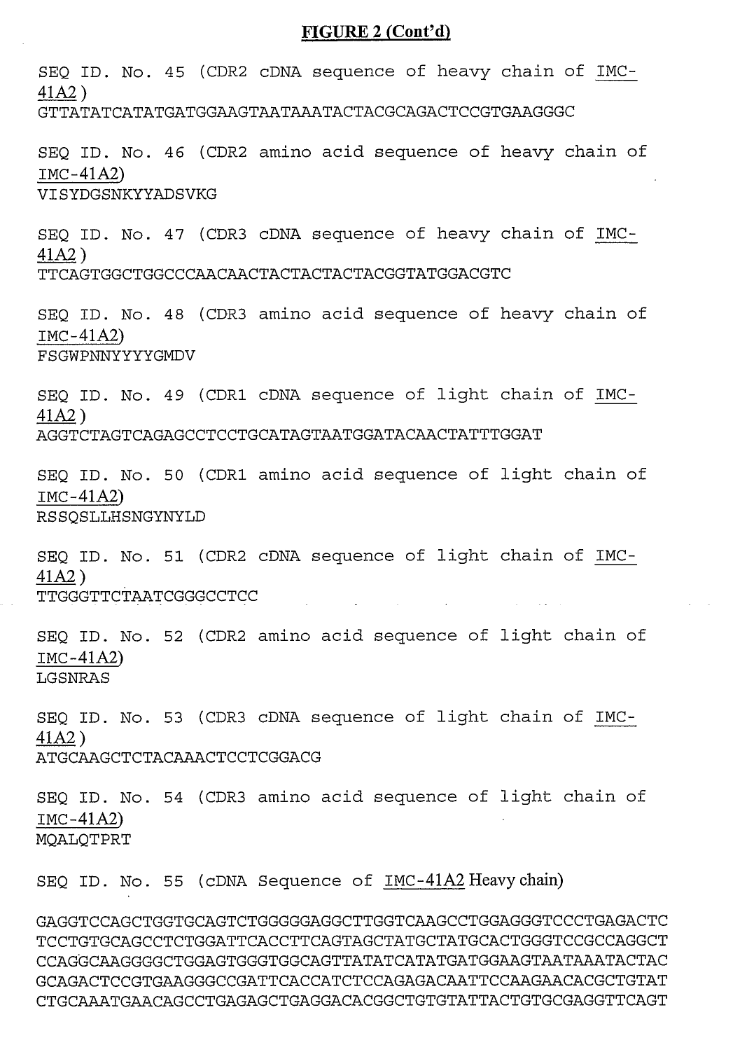

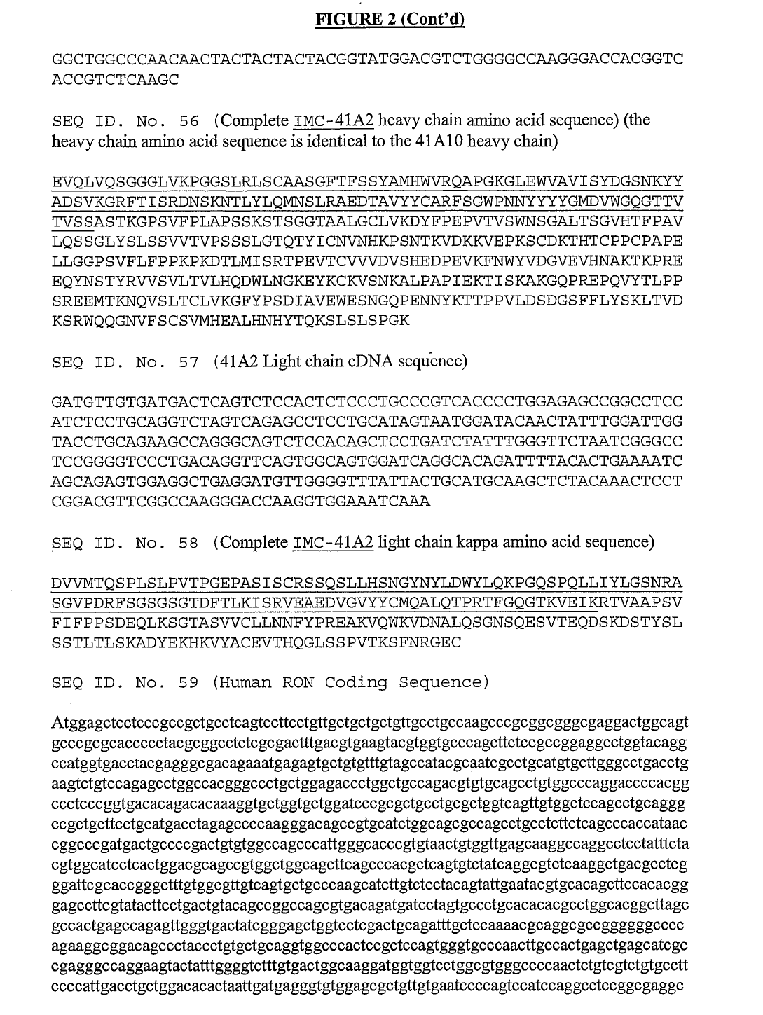

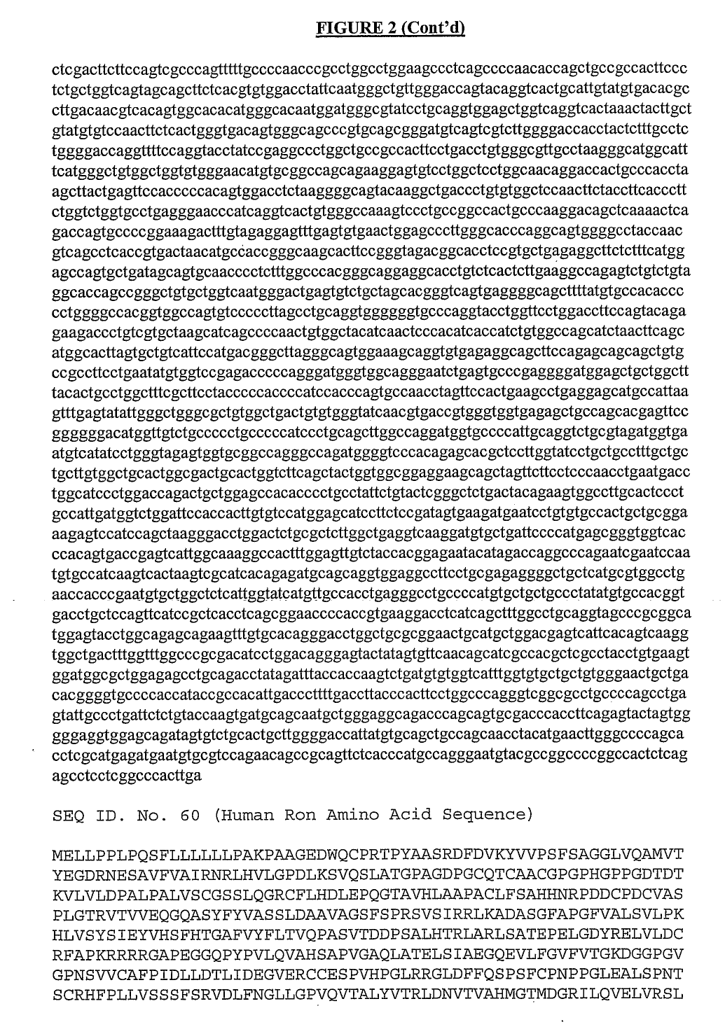

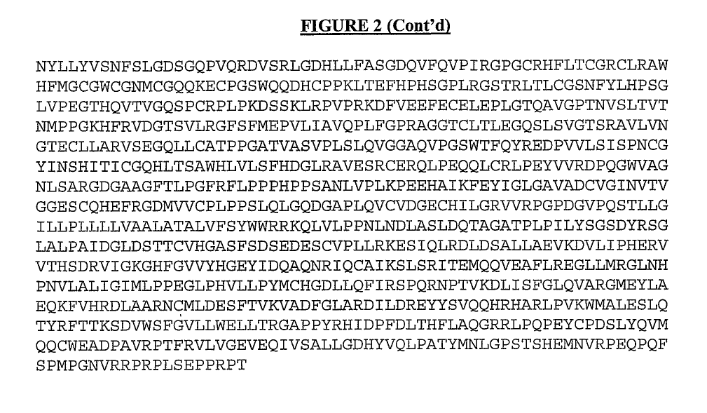

In a preferred embodiment, an antibody specific to RON having one, two, three, four, five, or all six complementarity-determining regions (CDRs) of the antibodies of the present invention is administered to a mammal. In one embodiment, the antibody administered has the variable regions of the antibodies of the present invention. FIG. 2 provides a summary of the sequences of the antibodies of the present invention. It is believed that IMC-41A2, IMC-41A10 and IMC-41B12 bind to the beta extracellular domain of RON, but such specificity may also arise by binding to other domains of RON, or binding to different epitopes in the same domain.

CDRs of antibodies isolated according to the present invention include:

Heavy Chain (IMC-41A2)

| | |
|---|---|
| CDR1H | SYAMH |
| CDR2H | VISYDGSNKYYADSVKG |
| CDR3H | FSGWPNNYYYYGMDV |

Light Chain (IMC-41A2)

| | |
|---|---|
| CDR1L | RSSQSLLHSNGYNYLD |
| CDR2L | LGSNRAS |
| CDR3L | MQALQTPRT |

Heavy Chain (IMC-41A10)

| | |
|---|---|
| CDR1 | SYAMH |
| CDR2 | VISYDGSNKYYADSVKG |
| CDR3 | FSGWPNNYYYYGMDV |

Light Chain (IMC-41A10)

| | |
|---|---|
| CDR1 | RSSQSLLHSNGFNYVD |
| CDR2 | FGSYRAS |
| CDR3 | MQALQTPPWT |

Heavy Chain (IMC-41B12)

| | |
|---|---|
| CDR1 | SHYWS |
| CDR2 | YIYYSGSTNYNPSLKS |
| CDR3 | IPNYYDRSGYYPGYWYFDL |

Light Chain (IMC-41B12)

| | |
|---|---|
| CDR1 | TLRSGFNVDSYRIS |
| CDR2 | YKSDSDK |
| CDR3 | MIWHSSAWV |

Variants of antibody and antibody fragments specific to RON also include polypeptides with amino acid sequences substantially similar to the amino acid sequence of the variable or hypervariable regions of the antibodies of the present invention. Substantially the same amino acid sequence is defined herein as a sequence with at least 70%, preferably at least about 80%, and more preferably at least about 90% homology to a compared amino acid sequence, as determined by the FASTA search method in accordance with Pearson and Lipman, Proc. Natl. Acad. Sci. USA 85, 2444-2448 (1988), including sequences that are at least about 70%, preferably at least about 80%, and more preferably at least about 90% identical. Such antibodies will have the same or similar binding, ligand blocking, and receptor inhibiting activities to antibodies of the invention that have substantially the same CDRs.

Variants of antibody and antibody fragments specific to RON also include antibodies having one or more conservative amino acid substitutions. A conservative amino acid substitution is defined as a change in the amino acid composition by way of changing one, two or more amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter peptide, polypeptide or protein characteristics (e.g., charge, isoelectric point, affinity, avidity, conformation, solubility) or activity. Typical substitutions that may be performed for such conservative amino acid substitution may be among the groups of amino acids as follows:

glycine (G), alanine (A), valine (V), leucine (L) and isoleucine (I);

aspartic acid (D) and glutamic acid (E);

alanine (A), serine (S) and threonine (T);

histidine (H), lysine (K) and arginine (R):

asparagine (N) and glutamine (Q);

phenylalanine (F), tyrosine (Y) and tryptophan (W)

Conservative amino acid substitutions can be made in, e.g., regions flanking the hypervariable regions primarily responsible for the selective and/or specific binding characteristics of the molecule, as well as other parts of the molecule, e.g., variable heavy chain cassette.

Antibodies, or fragments thereof, also include those for which binding characteristics have been improved by direct mutation, methods of affinity maturation, phage display, or chain shuffling.

Affinity and specificity can be modified or improved by mutating CDR and/or FW residues and screening for antigen binding sites having the desired characteristics (see, e.g., Yang et al., J. Mol. Biol., (1995) 254: 392-403). One way is to randomize individual residues or combinations of residues so that in a population of, otherwise identical antigen binding sites, subsets of from two to twenty amino acids are found at particular positions. Alternatively, mutations can be induced over a range of residues by error prone PCR methods (see, e.g., Hawkins et al., J. Mol. Biol., (1992) 226: 889-96). In another example, phage display vectors containing heavy and light chain variable region genes can be propagated in mutator strains of E. coli (see, e.g., Low et al., J. Mol. Biol., (1996) 250: 359-68). These methods of mutagenesis are illustrative of the many methods known to one of skill in the art.

Another manner for increasing affinity of the antibodies of the present invention is to carry out chain shuffling, where the heavy or light chain are randomly paired with other heavy or light chains to prepare an antibody with higher affinity. The various CDRs of the antibodies may also be shuffled with the corresponding CDRs in other antibodies.

The present invention further provides for antibodies which binds specifically to the same RON epitope(s) as those bound by the IMC-14A2, IMC-14A10 and IMC-14B12 antibodies. Such antibodies may be identified by their ability to compete with IMC-14A2, IMC-14A10 and IMC-14B12 RON binding. These epitopes are present on the extracellular domain of RON.

Additionally, the present invention provides isolated polynucleotides encoding the present antibodies or fragments thereof as well as expression vectors comprising these polynucleotide sequences operably linked to an expression sequence. These nucleotides are listed in FIG. 2. Recombinant host cells comprising the expression vector which express the present antibodies or fragments thereof are also provided. Methods are also provided for producing antibodies or fragments thereof comprising culturing these cells under conditions permitting expression of the antibodies or fragments thereof. The antibodies or fragments thereof can then be purified from the cell or cell culture medium.

Variants of nucleotides listed in FIG. 2 include those that encode for an antibody or antibody fragment having the same function as the antibodies of the present invention, i.e., to blocking activation of RON. Such variants have a sequence that is at least about 70%, preferably at least about 80%, and more preferably at least about 90% identical.

The present invention also provides for antibody fusion proteins. These fusion proteins may be encoded by the nucleotide sequences of FIG. 2 cloned adjacent to nucleotide sequences encoding enzymes, florescent proteins, a polypeptide tag or luminescent marker.

The nucleotide sequences of the invention also include: (a) the antibody DNA sequences shown in FIG. 2; (b) any nucleotide sequence that (i) hybridizes to the nucleotide sequence set forth in (a) under stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at p. 2.10.3) and (ii) encodes antibody or antibody fragment having substantially the same functionality; and (c) any nucleotide sequence that hybridizes to a DNA sequence that encodes the antibody sequences shown in FIG. 2 under less stringent conditions, such as moderately stringent conditions, e.g., washing in 0.2×SSC/0.1% SDS at 42° C. (Ausubel et al., 1989 supra), yet which still encodes an antibody or antibody fragment having substantially the same functionality. The functionality of the antibodies of the present invention is to block activation of RON.

The present invention also provides an expression vector containing a nucleic acid encoding an antibody of the present invention, or fragment thereof, operably linked to a control sequence, as well as a host cell containing such an expression vector. These host cells can be cultured under specific conditions permitting expression of antibodies of the present invention, or fragments thereof, and the antibodies then can be purified from the host cells.

Standard recombinant techniques and known expression vectors are used to express the antibodies of the invention. Vectors for expressing proteins in bacteria, especially E. Coli, are known. Such vectors include the PATH vectors described by Dieckmann and Tza goloff in J. Biol. Chem. 260, 1513-1520 (1985). These vectors contain DNA sequences that encode anthranilate synthetase (TrpE) followed by a polylinker at the carboxy terminus. Other expression vector systems are based on beta-galactosidase (pEX); lambda P_{L}; maltose binding protein (pMAL); and glutathione S-transferase (pGST)-see Gene 67, 31 (1988) and Peptide Research 3, 167 (1990).

Vectors useful in yeast are available. A suitable example is the 2 □ plasmid. Suitable vectors for expression in mammalian cells are also known. Such vectors include well-known derivatives of SV-40, adenovirus, retrovirus-derived DNA sequences and shuttle vectors derived from combination of functional mammalian vectors, such as those described above, and functional plasmids and phage DNA.

Further eukaryotic expression vectors are known in the art (e.g., P. J. Southern and P. Berg, J. Mol. Appl. Genet. 1, 327-341 (1982); S. Subramani et al, Mol. Cell. Biol. 1, 854-864 (1981); R. J. Kaufmann and P. A. Sharp, “Amplification And Expression Of Sequences Cotransfected with A Modular Dihydrofolate Reductase Complementary DNA Gene,” J. Mol. Biol. 159, 601-621 (1982); R. J. Kaufmann and P. A. Sharp, “Amplification And Expression Of Sequences Cotransfected with A Modular Dihydrofolate Reductase Complementary DNA Gene,” J. Mol. Biol. 159, 601-664 (1982); S. I. Scahill et al, “Expression And Characterization Of the Product Of A Human Immune Interferon DNA Gene In Chinese Hamster Ovary Cells,” Proc. Natl. Acad. Sci. USA 80, 4654-4659 (1983); G. Urlaub and L. A. Chasin, Proc. Natl. Acad. Sci. USA 77, 4216-4220, (1980)).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alphamating factors, and promoters derived from polyoma, adenovirus, retrovirus, and simian virus, e.g., the early and late promoters or SV40, and other sequences known to control the expression of genes of prokaryotic or eukaryotic cells and their viruses or combination thereof.

Vectors containing the control signals and DNA to be expressed, such as that encoding antibodies of the invention, antibody fragments thereof, are inserted into a host cell for expression. Some useful expression host cells include well-known prokaryotic and eukaryotic cells. Some suitable prokaryotic hosts include, for example, E. coli, such as E. coli SG-936, E. coli HB101, E. coli W3110, E. coli X1776, E. coli X2282, E. coli DHI, and E. coli MRC1, Pseudomonas, Bacillus, such as Bacillus subtilis, and Streptomyces. Suitable eukaryotic cells include yeast and other fungi, insect, animal cells, such as COS cells, cell lines of lymphoid origin such as lymphoma, myeloma (e.g. NSO) and CHO cells, human cells and plant cells in tissue culture.

A method of producing an antibody comprising culturing the host cell comprising the vector comprising the nucleic acid sequence encoding for the antibodies of the invention under conditions permitting expression of the antibody. Following expression in a host cell maintained in a suitable medium, the polypeptide or peptide to be expressed, such as that encoding the antibodies of the invention, may be isolated from the medium, and purified by methods known in the art. If the polypeptide or peptide is not secreted into the culture medium, the host cells are lysed prior to isolation and purification. A purified antibody is one that has been identified and separated and/or recovered from a component of its natural environment. Contaminant components of its natural environment are materials, which would interfere with diagnostic or therapeutic uses for the antibody, and may include enzymes, hormones, and other proteinaceous or non-proteinaceous solutes, generally have been removed.

The monoclonal antibodies specific for RON that are secreted by the subclones may be isolated or purified from the culture medium or ascites fluid by conventional immunoglobulin purification procedures such as, for example protein A-Sepharose, hydrolyapatite chromatography, gel electrophoresis, dialysis, or affinity chromatography.

In another embodiment, an antibody specific to RON is produced by expressing a nucleic acid encoding the antibody in a transgenic animal, such that the antibody is expressed and can be recovered. For example, the antibody can be expressed in a tissue specific manner that facilitates recovery and purification. In one such embodiment, an antibody of the invention is expressed in the mammary gland for secretion during lactation. Transgenic animals, include but are not limited to mice, goat, and rabbit.

The present invention provides for pharmaceutical compositions comprising anti-RON antibodies. In one embodiment, the composition may comprise one or more of the three specific antibodies disclosed herein. It is understood that the anti-RON antibodies of the invention, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers can further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

Carrier as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as TWEEN®, polyethylene glycol (PEG), and PLURONICS®.

The active ingredients may also be entrapped in microcapsules prepared, for example, by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles, and nanocapsules) or in macroemulsions. The formulations to be used for in vivo administration must be sterile. This is readily accomplished by filtration through sterile filtration membranes. Sustained-release preparations maybe prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization maybe achieved by modifying sulffiydryl residues, lyophilizing from acidic solutions, controlling moisture content, using appropriate additives, and developing specific polymer matrix compositions.

The present invention provides for method of treatments involving administration to a mammal in need thereof a therapeutically effective amount of antibodies or fragments thereof specific to RON. Preferably the mammal is a human. Such antibodies may include chimeric, humanized, murine, rabbit and human antibodies, obtained by various techniques. Preferred antibodies are those having specificity for an epitope on the extracellular domain of RON, including extracellular domains having deletions or other mutations. Preferably the antibody administered is a human antibody, more preferably having at least a single CDR sequence of IMC-41A10, IMC-41B12 or IMC-41A2. Conditions for which these methods are useful include tumors that express RON, inflammatory diseases, hyperproliferative diseases, and diseases of the liver, biliary tract, bile ducts, gall bladder and related hepatobiliary system.

Treatment means any treatment of a disease in an animal and includes: (1) preventing the disease from occurring in a mammal which may be predisposed to the disease but does not yet experience or display symptoms of the disease; e.g., prevention of the outbreak of the clinical symptoms; (2) inhibiting the disease, e.g., arresting its development; or (3) relieving the disease, e.g., causing regression of the symptoms of the disease.

In the methods of the present invention, a therapeutically effective amount of an antibody of the invention is administered to a mammal in need thereof. The term administering as used herein means delivering the antibodies of the present invention to a mammal by any method that can achieve the result sought. They can be administered, for example, intravenously or intramuscularly. Although human antibodies of the invention are particularly useful for administration to humans, they can be administered to other mammals as well. The term mammal as used herein is intended to include, but is not limited to, humans, laboratory animals, domestic pets and farm animals. Therapeutically effective amount means an amount of antibody of the present invention that, when administered to a mammal, is effective in producing the desired therapeutic effect, such as inhibiting kinase activity or inhibition of tumor growth.

The present anti-RON antibodies can be administered for therapeutic treatments to a patient suffering from a tumor or angiogenesis associated pathologic condition in an amount sufficient to prevent, inhibit, or reduce the progression of the tumor or pathologic condition. Progression includes, e.g., the growth, invasiveness, metastases and/or recurrence of the tumor or pathologic condition. An amount adequate to accomplish this is defined as a therapeutically effective dose. Amounts effective for this use will depend upon the severity of the disease and the general state of the patient's own immune system. Dosing schedules will also vary with the disease state and status of the patient, and will typically range from a single bolus dosage or continuous infusion to multiple administrations per day (e.g., every 4-6 hours), or as indicated by the treating physician and the patient's condition. It should be noted, however, that the present invention is not limited to any particular dose.

A suitable dose for the antibodies of the present invention may be determined based on the in vivo data illustrated in the present invention. The in vivo experiment used a dose of about 1 mg/20 grams every three days. The average mouse is about 0.02 Kg and its volume is about 0.008 m². The average human is about 70 Kg, and its volume is about 1.85 m². A dose of about 200 mg/m² corresponds to about 40 mg/Kg into a mouse, which is roughly about 2.6 mg/Kg in a human. To put this dose in perspective, another antibody, Erbitux®, is administered at 1 dose pre week of about 250 mg/m², which is about 6.5 mg/Kg in human. Based on these calculations and experiments, the dose administered to a human is preferably about 1 to about 10 mg/Kg, more preferably about 3 to about 8 mg/Kg (1 dose per week). The dose might be similar to that for Erbitux®, about 6 to about 7 mg/Kg.

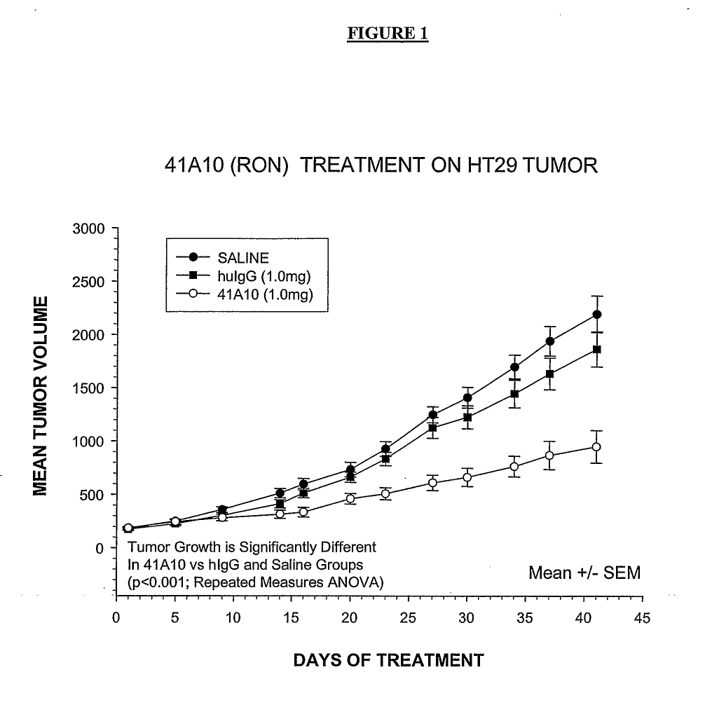

The present invention for the first time demonstrates that in vivo inhibition of RON with an antibody that inhibits tumor growth. A RON antibody inhibits HT-29 cells grown sub-cutaneously in nude mice. Preferably, the tumor growth is suppressed at least about 20%, more preferably at least about 40%. FIG. 1 shows about a 50-60% decrease in HT-29 tumor growth over a 40-day period.

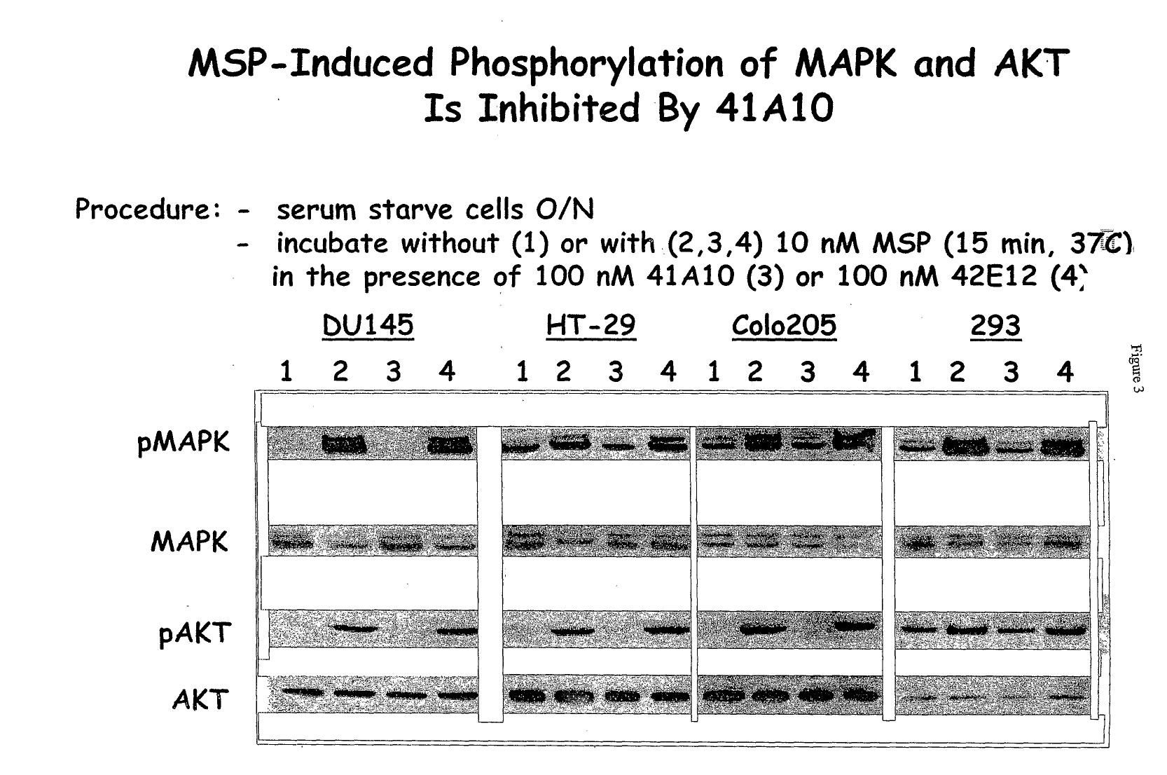

RON antibodies can block, preferably at least about 60%, more preferably about 80%, and most preferably about 100%, MSP-induced phosphorylation of RON, MAPK, and AKT (ex. HT-29, Colo205, AGS and DU145). In FIG. 3, the bands for Lane 1 and 3 are almost identical, pointing to such complete blocking of phosphorylation. Phosphorylation of MAPK and AKT are considered important for cell proliferation (increase in cell number overtime), migration (movement of cells towards an agent, particularly MSP, i.e., chemo-attraction), invasion (ability to move through a new tissue) and survival respectively. The proliferation of adherent HT-29 and Colo205 cells are preferably inhibited about 20% to about 30%, more preferably about 25% in the presence of a RON antibody and 10% serum. In addition, when HT-29 and Colo205 are grown in soft agar in the presence of A RON antibody and 10% serum, colony formation is preferably inhibited about 60% to about 80%, more preferably about 75% for HT-29, and about 50% to about 70%, more preferably 60% for Colo205.

The present invention is based on the observation that RON specific antibodies can inhibit growth of cancer cells in soft agar and inhibit proliferation while growing as adherent cells in cell culture conditions. A RON antibody can significantly retard the ability of the cancer cell line to form tumors when injected into nude mice, which demonstrates that inhibition of the RON receptor tyrosine kinase negatively influences the proliferation of colon cancer cells.

Using conventional Western blot and flow cytometry procedures, it has been found that RON is expressed in many human tumor cell lines: Colon (HT-29, Colo205, HCT-116, DLD-1, Sw480, Sw620), Pancreatic (BXPC-3, CAPAN-2, ASPC-1, HPAF-11, L3.7p1#7, Hs766T), Prostate (DU-145, PC-3), Stomach (AGS, NCI-N87), Lung (A549, H596) and Liver (HepG2, SNU-182). Accordingly tumors derived from a variety of cell types are therapeutic targets for a RON antibody.

Tumors to be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued.

Tumors that can be treated include tumors that are not vascularized, or not yet substantially vascularized, as well as vascularized tumors. Examples of solid tumors, which can be accordingly treated, include breast carcinoma, lung carcinoma, colorectal carcinoma, pancreatic carcinoma, glioma and lymphoma. Some examples of such tumors include epidermoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors. Other examples include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma. Of particular interest are colon, pancreatic, prostate, stomach, lung and liver cancers.

Accordingly, the human anti-RON antibodies may be effective for treating subjects with vascularized tumors or neoplasms or angiogenic diseases. Such tumors and neoplasms include, for example, malignant tumors and neoplasms, such as blastomas, carcinomas or sarcomas, and highly vascular tumors and neoplasms. Cancers that may be treated by the methods of the present invention include, for example, cancers of the brain, genitourinary tract, lymphatic system, stomach, renal, colon, larynx and lung and bone. Non-limiting examples further include epidermoid tumors, squamous tumors, such as head and neck tumors, colorectal tumors, prostate tumors, breast tumors, lung tumors, including lung adenocarcinoma and small cell and non-small cell lung tumors, pancreatic tumors, thyroid tumors, ovarian tumors, and liver tumors. The method is also used for treatment of vascularized skin cancers, including squamous cell carcinoma, basal cell carcinoma, and skin cancers that can be treated by suppressing the growth of malignant keratinocytes, such as human malignant keratinocytes. Other cancers that may be treated include Kaposi's sarcoma, CNS neoplasms (neuroblastomas, capillary hemangioblastomas, meningiomas and cerebral metastases), melanoma, gastrointestinal and renal carcinomas and sarcomas, rhabdomyosarcoma, glioblastoma, including glioblastoma multiforme, and leiomyosarcoma.

In another aspect of the invention, the anti-RON antibodies inhibit tumor-associated angiogenesis. Stimulation of vascular endothelium by Receptor Tyrosine Kinases is associated with vascularization of tumors. Typically, vascular endothelium is stimulated in a paracrine fashion.

Antineoplastic agents, may be administered separately or as a conjugate to the antibody RON. The anti-neoplastic agents which are presently known in the art or being evaluated can be grouped into a variety of classes including, for example, mitotic inhibitors, alkylating agents, anti-metabolites, intercalating antibiotics, growth factor inhibitors, cell cycle inhibitors, enzymes, topoisomerase inhibitors, anti survival agents, biological response modifiers, anti-hormones, and anti-angiogenesis agents.

Many of the known antineoplastic agents are small organic molecules. Embodiments of the invention include methods in which a topoisomerase inhibitor is administered in combination with an antibody that binds to RON. The inhibitors can be inhibitors of topoisomerase I or topoisomerase II. Topoisomerase I inhibitors include irinotecan (CPT-11), aminocamptothecin, camptothecin, DX-8951f, topotecan. Topoisomerase II inhibitors include etoposide (VP-16), and teniposide (VM-26). Other substances are currently being evaluated with respect to topoisomerase inhibitory activity and effectiveness as anti-neoplastic agents. The anti-neoplastic agent can be an alkylating agent or an anti-metabolite. Examples of alkylating agents include, but are not limited to, cisplatin, cyclophosphamide, melphalan, and dacarbazine. Additional small organic molecules include cytotoxic and/or chemotherapeutic agents such as taxol, doxorubicin, actinomycin-D, methotrexate, gemcitabine, oxyplatin, fluorouracil (5-FU), leucourin (LU), cisplatin, paclitaxel, docetaxel, vinblastine, epothilone, cisplatin/carboplatin and Pegylated adriamycin. The small organic molecules may be administered in combinations such as: (CPT-11; 5-FU; LU); (Paclitaxel; 5-FU); and (CPT-11; 5-FU; LU).

The anti-neoplastic agent also includes radiation. When the anti-neoplastic agent is radiation, the source of the radiation can be either external (external beam radiation therapy—EBRT) or internal (brachytherapy—BT) to the patient being treated. The dose of anti-neoplastic agent administered depends on numerous factors, including, for example, the type of agent, the type and severity tumor being treated and the route of administration of the agent. It should be emphasized, however, that the present invention is not limited to any particular dose. Radiation may be used in conjunction with other antineoplastic agents.

In another aspect of the invention, anti-RON antibodies or antibody fragments can be chemically or biosynthetically linked to anti-tumor agents or detectable signal-producing agents, particularly when the antibody is internalized. Anti-tumor agents linked to an antibody include any agents which destroy or damage a tumor to which the antibody has bound or in the environment of the cell to which the antibody has bound. For example, an anti-tumor agent is a toxic agent such as a chemotherapeutic agent or a radioisotope. Suitable chemotherapeutic agents are known to those skilled in the art and include anthracyclines (e.g. daunomycin and doxorubicin), methotrexate, vindesine, neocarzinostatin, cis-platinum, chlorambucil, cytosine arabinoside, 5-fluorouridine, melphalan, ricin and calicheamicin. The chemotherapeutic agents are conjugated to the antibody using conventional methods (See, e.g., Hermentin and Seiler, Behring Inst. Mitt. 82:197-215 (1988)).

The RON antibody may also be administered with radioisotopes to a cancer patient. Suitable radioisotopes for use as anti-tumor agents are also known to those skilled in the art. For example, 131I or 211At is used. These isotopes are attached to the antibody using conventional techniques (See, e.g., Pedley et al., Br. J. Cancer 68, 69-73 (1993)). Alternatively, the anti-tumor agent which is attached to the antibody is an enzyme which activates a prodrug. In this way, a prodrug is administered which remains in its inactive form until it reaches the tumor site where it is converted to its cytotoxin form once the antibody complex is administered. In practice, the antibody-enzyme conjugate is administered to the patient and allowed to localize in the region of the tissue to be treated. The prodrug is then administered to the patient so that conversion to the cytotoxic drug occurs in the region of the tissue to be treated. Alternatively, the anti-tumor agent conjugated to the antibody is a cytokine such as interleukin-2 (IL-2), interleukin-4 (IL-4) or tumor necrosis factor alpha (TNF-α). The antibody targets the cytokine to the tumor so that the cytokine mediates damage to or destruction of the tumor without affecting other tissues. The cytokine is fused to the antibody at the DNA level using conventional recombinant DNA techniques. Interferons may also be used

The present invention also provides a method of treating a non-cancer hyperproliferative disease in a mammal comprising administering to the mammal an effective amount of the antibody of the present invention. As disclosed herein, “hyperproliferative disease” is defined as a condition caused by excessive growth of non-cancer cells that express a member of the RON family of receptors. The excess cells generated by a hyperproliferative disease express RON at normal levels or they may overexpress RON.

The types of hyperproliferative diseases that can be treated in accordance with the invention are any hyperproliferative diseases that are stimulated by a ligand of RON or mutants of such ligands. Examples of hyperproliferative disease include psoriasis, actinic keratoses, and seborrheic keratoses, warts, keloid scars, and eczema. Also included are hyperproliferative diseases caused by virus infections, such as papilloma virus infection. For example, psoriasis comes in many different variations and degrees of severity. Different types of psoriasis display characteristics such as pus-like blisters (pustular psoriasis), severe sloughing of the skin (erythrodermic psoriasis), drop-like dots (guttae psoriasis) and smooth inflamed lesions (inverse psoriasis). The treatment of all types of psoriasis (e.g., psoriasis vulgaris, psoriasis pustulosa, psoriasis erythrodermica, psoriasis arthropathica, parapsoriasis, palmoplantar pustulosis) is contemplated by the invention.

For treatment of hyperproliferative disease, administration of the antibodies of the invention as described above can be combined with administration of any conventional treatment agent. For example, when the hyperproliferative disease is psoriasis, there are a variety of conventional systemic and topical agents available. Systemic agents for psoriasis include methotrexate, and oral retinoids, such as acitretin, etretinate, and isotretinoin. Other systemic treatments of psoriasis include hydroxyurea, NSAIDS, sulfasalazine, and 6-thioguanine. Antibiotics and antimicrobials can be used to treat or prevent infection that can cause psoriasis to flare and worsen. Topical agents for psoriasis include anthralin, calcipotriene, coal tar, corticosteroids, retinoids, keratolytics, and tazarotene. Topical steroids are one of the most common therapies prescribed for mild to moderate psoriasis. Topical steroids are applied to the surface of the skin, but some are injected into the psoriasis lesions.

Hyperproliferative disease treatments further include administration of anti-RON antibodies in combination with phototherapy. Phototherapy includes administration of any wavelength of light that reduces symptoms of the hyperproliferative disease, as well as photoactivation of a chemotherapeutic agent (photochemotherapy). For further discussion of treatment of hyperproliferative disorders, see WO 02/11677 (Teufel et al.) (Treatment of hyperproliferative diseases with epidermal growth factor receptor antagonists).

In the present invention, any suitable method or route can be used to administer anti-RON antibodies of the invention, and optionally, to co-administer anti-neoplastic agents and/or antagonists of other receptors. The anti-neoplastic agent regimens utilized according to the invention, include any regimen believed to be optimally suitable for the treatment of the patient's neoplastic condition. Different malignancies can require use of specific anti-tumor antibodies and specific anti-neoplastic agents, which will be determined on a patient to patient basis. Routes of administration include, for example, oral, intravenous, intraperitoneal, subcutaneous, or intramuscular administration. The dose of antagonist administered depends on numerous factors, including, for example, the type of antagonists, the type and severity tumor being treated and the route of administration of the antagonists. It should be emphasized, however, that the present invention is not limited to any particular method or route of administration.

The anti-RON antibodies, particularly for treatment of cancers, can also be administered with intracellular RTK antagonists that inhibit activity of RTKs or their associated downstream signaling elements that are involved in tumor growth or tumor-associated angiogenesis. The intracellular RTK antagonists are preferably small molecules. Some examples of small molecules include organic compounds, organometallic compounds, salts of organic compounds and organometallic compounds, and inorganic compounds. Atoms in a small molecule are linked together via covalent and ionic bonds; the former is typical for small organic compounds such as small molecule tyrosine kinase inhibitors and the latter is typical of small inorganic compounds. The arrangement of atoms in a small organic molecule may represent a chain, e.g. a carbon-carbon chain or carbon-heteroatom chain or may represent a ring containing carbon atoms, e.g. benzene or a policyclic system, or a combination of carbon and heteroatoms, i.e., heterocycles such as a pyrimidine or quinazoline. Although small molecules can have any molecular weight they generally include molecules that would otherwise be considered biological molecules, except their molecular weight is not greater than 650 D. Small molecules include both compounds found in nature, such as hormones, neurotransmitters, nucleotides, amino acids, sugars, lipids, and their derivatives as well as compounds made synthetically, either by traditional organic synthesis, bio-mediated synthesis, or a combination thereof. See e.g. Ganesan, Drug Doscov. Today 7(1): 47-55 (January 2002); Lou, Drug Discov. Today, 6(24): 1288-1294 (December 2001).

More preferably, the small molecule to be used as an intracellular RTK antagonist according to the present invention is an intracellular RON antagonist that competes with ATP for binding to EGFR's intracellular binding region having a kinase domain or to proteins involved in the signal transduction pathways of EGFR activation. Examples of such signal transduction pathways include the ras-mitogen activated protein kinase (MAPK) pathway, the phosphatidylinosital-3 kinase (Pl3K)-Akt pathway, the stress-activated protein kinase (SAPK) pathway, and the signal transducers and activators of transcription (STAT) pathways. Non-limiting examples of proteins involved in such pathways (and to which a small molecule RON antagonist according to the present invention can bind) include GRB-2, SOS, Ras, Raf, MEK, MAPK, and matrix metalloproteinases (MMPs).

The method of treatment described herein, particularly for cancers, may also be carried out with administration of other antibodies. For example, an antibody against EGFR, such as Erbitux® (cetuximab), may also be administered, particularly when treating colon cancer. Erbitux® MAb is a recombinant, human/mouse chimeric, monoclonal antibody that binds specifically to the extracellular domain of the human EGFR. Erbitux® is an EGFR antagonist, which blocks ligand binding to EGFR, prevents receptor activation, and inhibits growth of tumor cells that express EGFR. Erbitux® has been approved for use in combination with or without irinotecan in the treatment of patients with epidermal growth factor receptor-expressing, metastatic colorectal cancer who are refractory or can not tolerate irinotecan-based chemotherapy. Erbitux® has also been shown to be effective for treatment of psoriasis.

Other antibodies for combination use include Herceptin® (trastuzumab) (against breast cancer cells that express HER2, or HER2 expression on other cancer cells) and Avastino (bevacizumab) (antibodies that inhibit angiogenesis). Other antibodies are 2F8 and A12, specific to IGFR, which have the following CDR sequences:

Heavy Chain (2F8/A12)

| | |
|---|---|
| CDR1 | SYALS |
| CDR2 | GIIPWGTANYAQKFQG |
| CDR3 | APLRFLEWSTQDHYYYYYM |
| | DV |

Light Chain (2F8)

| | |
|---|---|
| CDR1 | QGDSLRSYYAS |
| CDR2 | GKNNRPS |
| CDR3 | NSRDNSDNRLI |

Light Chain (A12)

| | |
|---|---|
| CDR1 | QGDSLRSYYAT |
| CDR2 | GENKRPS |
| CDR3 | KSRDGSGQHLV |

The method of treatment described herein may also be carried out with administration of other peptides. For example, variants of MSP may be administered where the variants bind to RON but do not activate RON, or at least competitively inhibit MSP. See e.g. U.S. Publ. No. 2003/0073656

The administration of the RON antibodies with other antibodies and/or small organic molecules may occur simultaneously, or separately, via the same or different route.

Anti-RON antibodies of the invention can be administered with RON antagonists, and/or antagonists of other RTKs, such as antibodies that block RTK ligands or otherwise inhibit the RTKs. An example of other such RTKs include EGFR, c-met and VEGFR.

In one embodiment of the present invention, an anti-RON antibody is used in combination with a VEGFR antagonist. In one embodiment of the invention, an anti-RON antibody is used in combination with a receptor antagonist that binds specifically to VEGFR-2/KDR receptor (PCT/US92/01300, filed Feb. 20, 1992; Terman et al., Oncogene 6: 1677-1683 (1991)). In another embodiment, an anti-RON antibody is used in combination with a receptor antagonist that binds specifically to VEGFR-1/Flt-1 receptor (Shibuya M. et al., Oncogene 5, 519-524 (1990)). Particularly preferred are antigen-binding proteins that bind to the extracellular domain of VEGFR-1 or VEGFR-2 and block binding by ligand (VEGF or PlGF), and/or inhibit VEGF-induced or PlGF-induced activation. For example, Mab IMC-1121 binds to soluble and cell surface-expressed KDR. Mab IMC-1121 comprises the V_{H} and V_{L} domains obtained from a human Fab phage display library. (See WO 03/075840) In another example, ScFv 6.12 binds to soluble and cell surface-expressed Flt-1. ScFv 6.12 comprises the V_{H} and V_{L} domains of mouse monoclonal antibody MAb 6.12. A hybridoma cell line producing MAb 6.12 has been deposited as ATCC number PTA-3344.

Another example of such an RTK is insulin-like growth factor receptor (IGFR). In certain tumor cells, inhibition of RTK function can be compensated by upregulation of other growth factor receptor signaling pathways, and particularly by RON stimulation. Further, inhibition of IGFR signaling results in increased sensitivity of tumor cells to certain therapeutic agents. Stimulation of either RON or IGFR results in phosphorylation of common downstream signal transduction molecules, including Akt and p44/42, although to different extents. Accordingly, in an embodiment of the invention, an IGFR antagonist (e.g., an antibody that binds to IGF or IGFR and inhibits the receptor) is coadministered with an antibody of the invention, thereby blocking a second input into the common downstream signaling pathway (e.g., inhibiting activation of Akt and/or p44/42). An example of a human antibody specific for IGFR is IMC-A12 (See WO 2005/016970).

Another receptor that may be targeted in combination with RON is EGFR. EGFR may be targeted with an antibody such as Erbitux® as described above, or with a small organic molecule. One example of a small molecule RTK antagonist is IRESSA™ (ZD1939), which is a quinozaline derivative that functions as an ATP-mimetic to inhibit EGFR. See U.S. Pat. No. 5,616,582 (Zeneca Limited); WO 96/33980 (Zeneca Limited) at p. 4; see also, Rowinsky et al., Abstract 5 presented at the 37th Annual Meeting of ASCO, San Francisco, Calif., 12-15 May 2001; Anido et al., Abstract 1712 presented at the 37th Annual Meeting of ASCO, San Francisco, Calif., 12-15 May 2001. Another examples of a small molecule EGFR antagonist is TARCEVA™ (OSI-774), which is a 4-(substituted phenylamino)quinozaline derivative [6,7-Bis(2-methoxy-ethoxy)-quinazolin-4-yl]-(3-ethynyl-phenyl)amine hydrochloride] EGFR inhibitor. See WO 96/30347 (Pfizer Inc.) at, for example, page 2, line 12 through page 4, line 34 and page 19, lines 14-17. See also Moyer et al., Cancer Res., 57: 4838-48 (1997); Pollack et al., J. Pharmacol., 291: 739-48 (1999). TARCEVA™ may function by inhibiting phosphorylation of EGFR and its downstream PI3/Akt and MAP (mitogen activated protein) kinase signal transduction pathways resulting in p27-mediated cell-cycle arrest. See Hidalgo et al., Abstract 281 presented at the 37th Annual Meeting of ASCO, San Francisco, Calif., 12-15 May 2001. The above small organic molecules may also inhibit RON.

Other examples of growth factor receptors involved in tumorgenesis are the receptors for platelet-derived growth factor (PDGF), nerve growth factor (NGF), and fibroblast growth factor (FGF). These receptors may be targeted in combination with RON.

In another embodiment, the RON antagonist can be administered in combination with one or more suitable adjuvants, such as, for example, cytokines (IL-10 and IL-13, for example) or other immune stimulators, such as, but not limited to, chemokine, tumor-associated antigens, and peptides.

In a combination therapy, the anti-RON antibody is administered before, during, or after commencing therapy with another agent, as well as any combination thereof, i.e., before and during, before and after, during and after, or before, during and after commencing the anti-neoplastic agent therapy. For example, the anti-RON antibody can be administered between 1 and 30 days, preferably 3 and 20 days, more preferably between 5 and 12 days before commencing radiation therapy. In a preferred embodiment of the invention, chemotherapy is administered concurrently with or, more preferably, subsequent to antibody therapy.

The invention further contemplates RON antibodies or antibody fragments of the invention to which target or reporter moieties are linked. Target moieties are first members of binding pairs. Anti-tumor agents, for example, are conjugated to second members of such pairs and are thereby directed to the site where the antigen-binding protein is bound. A common example of such a binding pair is avidin and biotin. In a preferred embodiment, biotin is conjugated to an antigen-binding protein of the invention, and thereby provides a target for an anti-tumor agent or other moiety which is conjugated to avidin or streptavidin. Alternatively, biotin or another such moiety is linked to an antigen-binding protein of the invention and used as a reporter, for example in a diagnostic system where a detectable signal-producing agent is conjugated to avidin or streptavidin.

Detectable signal-producing agents are useful in vivo and in vitro for diagnostic purposes. The signal producing agent produces a measurable signal which is detectable by external means, usually the measurement of electromagnetic radiation. For the most part, the signal producing agent is an enzyme or chromophore, or emits light by fluorescence, phosphorescence or chemiluminescence. Chromophores include dyes which absorb light in the ultraviolet or visible region, and can be substrates or degradation products of enzyme catalyzed reactions.

Moreover, included within the scope of the present invention is use of the present antibodies in vivo and in vitro for investigative or diagnostic methods, which are well known in the art. The diagnostic methods include kits, which contain antibodies of the present invention. Such kits might be useful for identification of individuals at risk for certain type of cancers by detecting over-expression of RON on cells of such individuals. Additionally, the antibodies of the present invention may be used in the laboratory for research due to their ability to identify RON.

The present invention also includes kits for inhibiting tumor growth and/or tumor-associated angiogenesis comprising a therapeutically effective amount of a human anti-EGFR antibody. The kits can further contain any suitable antagonist of, for example, another growth factor receptor involved in tumorigenesis or angiogenesis (e.g., VEGFR-1/Flt-1, VEGFR-2, PDGFR, IGFR, NGFR, EGFR, FGFR, etc, as described above). Alternatively, or in addition, the kits of the present invention can further comprise an anti-neoplastic agent. Examples of suitable anti-neoplastic agents in the context of the present invention have been described herein. The kits of the present invention can further comprise an adjuvant; examples have also been described above.

The present invention further provides the method of identifying and isolating antibodies having the same functionality of IMC-41A2, IMC-41A10 or IMC-41B12, or fragments thereof, wherein the screening of the library includes providing an affinity matrix having RON containing ligand binding function bound to a solid support, contacting the affinity matrix with the library of antibody fragments, and separating the antibody fragments that bind to the affinity matrix from the antibody fragments that do not bind the affinity matrix.

By solid support is meant a non-aqueous matrix to which the RON can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

All patents and literature references cited in the present specification are hereby incorporated by reference in their entirety.

### EXAMPLES

The following examples are offered for illustrative purposes only, and are not intended to limit the scope of the present invention in any way. The examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989) Molecular Cloning: A laboratory Manual, 2^{nd} Edition, Cold Spring Harbor Laboratory Press.

Materials and Methods

### Development and Characteristics of Two Fab Anti-RON Antibodies

### (IMC-41A10 and IMC-41B12)

Selection of human anti-RON Fab antibodies from a phage display library. A large human Fab phage display library containing 3.7×10¹⁰ clones was used for the selection. The library stock was grown to log phase, rescued with M13K07 helper phage and amplified overnight in 2YTAK medium (2YT containing 100 μg/ml of ampicillin and 50 g/ml of kanamycin) at 30° C. The phage preparation was precipitated in 4% PEG/0.5M NaCl, resuspended in 3% fat-free milk/PBS containing 500 μg/ml of Fc protein and incubated at 37° C. for 1 h to capture phage displaying anti-Fc Fab fragments and to block other nonspecific binding.

RON-Fc (10 μg/ml in PBS; Sigma-Aldrich) coated Maxisorp Star tubes (Nunc, Rosklide, Denmark) were first blocked with 3% milk/PBS at 37° C. for 1 h, and then incubated with the phage preparation at RT for 1 h. The tubes were washed 20 times with PBST (PBS containing 0.1% Tween-20) followed by 20 washes with PBS. The bound phage was eluted at RT for 10 min with 1 ml of a freshly prepared solution of 100 mM triethylamine (Sigma, St. Louis, Mo.). Phage were eluted with 100 mM triethylamine and neutralized with Tris.HCl, pH7.4 and used to re-infect incubated with 10 ml of mid-log phase TG1 cells at 37° C. for 30 min without shaking followed by a 30 min shake. The infected TG1 cells were pelleted and plated onto several large 2YTAG plates and incubated overnight at 30° C. All the colonies grown on the plates were scraped into 3 to 5 ml of 2YTA medium, mixed with glycerol (final concentration: 10%), aliquoted and stored at −70° C. For the next round of selection, 100 μl of the phage stock was added to 25 ml of 2YTAG medium and grown to mid-log phase. The culture was rescued with M13K07 helper phage, amplified, precipitated, and used for selection following the procedure described above, with reduced concentrations of RON-Fc immobilized on the immunotube and increased number of washes after the binding process.

ELISA to Detect Phage Fab Antibodies from Phage that Bind to RON. Individual TG1 clones were picked and grown at 37° C. in 96 well plates and rescued with M13K07 helper phage as described above. The amplified phage preparation was blocked with 1/6 volume of 18% milk/PBS at RT for 1 h and 100 μl/well was added to Maxi-sorp 96-well microtiter plates (Nunc) coated with RON-Fc or Fc (1 μg/ml×100 μl). After incubation at RT for 1.5 h the plates were washed 3 times with PBST and incubated with a 1:5000 dilution of a mouse anti-M13 phage-HRP conjugate (Amersham Pharmacia Biotech, Piscataway, N.J.). The plates were washed 5 times, TMB peroxidase substrate (KPL, Gaithersburg, Md.) added, and the absorbance at 450 nm read using a microplate reader (Molecular Device, Sunnyvale, Calif.).

Expression and purification of the soluble Fab fragments. Plasmids of individual clones were used to transform a nonsuppressor Escherichia coli host HB2151. Expression of the Fab fragments in HB2151 was induced by culturing the cells in 2YTA medium containing 1 mM isopropyl-1-thio-D-galactopyranoside (Sigma) at 30° C. A periplasmic extract of the cells was prepared by resuspending the cell pellet in 25 mM Tris (pH 7.5) containing 20% (w/v) sucrose, 200 mM NaCl, 1 mM EDTA and 0.1 mM phenylmethylsulfonyl fluoride (PMSF), followed by incubation at 4° C. with gentle shaking for 1 hr. After centrifugation at 15,000 rpm for 15 min, the soluble Fab protein was purified from the supernatant by affinity chromatography using a Protein G column following the manufacturer's protocol (Amersham Pharmacia Biotech).

ELISA to Detect Fab Antibodies That Block the MSP/RON Interaction. Maxi-sorp 96-well microtiter plates (Nunc) were coated with (1 μg/ml×100 μl) MSP (R&D Systems) at RT for 1.5 hours. After washing the wells, they were blocked with 3% PBS/milk. Anti-RON phage antibodies that were converted to Fab or full IgG were pre-incubated with RON-Fc (25 ng/well) at RT for 1 hour. The Fab/RON-Fc or IgG/RON-Fc mixtures were then added to the MSP-coated wells and allowed to incubate for 1.5 h at RT. After several washes, a 1:1000 dilution of the anti-human IgG, Fab-specific-HRP conjugated antibody was added to the plates for 1.5 h at RT in order to detect the anti-RON Fab or IgG that bound to RON, but that did not block the MSP/RON interaction.

DNA BstN I pattern analysis and nucleotide sequencing. The diversity of the anti-RON phage Fab clones after each round of selection was analyzed by restriction enzyme digestion patterns (i.e., DNA fingerprints). The Fab gene insert of individual clones was PCR amplified using primers: PUC19 reverse, 5′ AGCGGATAACAATTTCACACAGG 3′; and fdtet seq, 5′ GTCGTCTTTCCAGACGTTAGT 3′. The amplified product was digested with a frequent-cutting enzyme, BstN I, and analyzed on a 3% agarose gel. DNA sequences of representative clones from each digestion pattern were determined by dideoxynucleotide sequencing.

Cloning of Fab Heavy and Light Chain Fragments to Produce IgG Antibodies. The DNA sequences encoding the heavy and light chain genes from the IMC-IMC-41A10 and IMC-41B12 Fab candidates were amplified by PCR for cloning into glutamine synthetase system expression vectors (Lonza Biologics plc, Slough, Berkshire, United Kingdom). The DNA and protein sequences for the variable regions of the IMC-41A10 and IMC-41B12 heavy and light chains are shown in FIG. 1. Engineered immunoglobulin expression vectors were stably transfected in NS0 cells using glutamine synthetase selection, and clones were screened for antibody expression by anti-Fc ELISA. Full-length IgG1 antibody was purified by protein A affinity chromatography (Poros A; PerSeptive Biosystems Inc., Foster City, Calif.).

BIAcore-Analysis. The binding kinetics of soluble Fab and antibody proteins to RON was determined by using a BIACORE 3000 (BIAcore, Piscataway, N.J.). Recombinant RON-Fc was immobilized onto a sensor chip, and Fab or antibody was injected at various concentrations. Sensorgrams were obtained and evaluated using BIA Evaluation 2.0 software to determine rate constants. The affinity constant, K_{D}, was calculated from the ratio of the rate constants K_{off}/Kₒₙ. The “Kon, M⁻¹.S⁻¹” and “Koff, S⁻¹” rates of the interaction were used to determine the affinity (Kd, M) of the antibody/receptor interaction. The Kd, Kon, and Koff rates for IMC-41A10 were 1.5e-9, 8.4e4 and 1.3e-4. For IMC-41B12, they were: 1e-10, 1.7e6 and 1.7e-4.

Flow Cytometry of RON Cell Surface Expression. One million cells from adherent cancer cell lines were incubated in PBS+5% FCS for 30 minutes with 5 micrograms IMC-41A10 at 4° C. After a wash in PBS+5% FCS, cells were incubated with anti-human IgG phycoerythrin-conjugated secondary antibody (Jackson Immuno Research) for 30 minutes at 4° C. After a PBS+5% FCS wash, cells were analyzed by flow cytometry using a FACSvantage SE flow cytometer (Becton Dickinson).

Western Blotting and Immunoprecipitation. Cells were plated into 10-cm or 6-well culture dishes and grown to 70-80% confluence. Monolayers were washed twice in PBS and cultured overnight in serum-free medium. Antibody was then added in fresh serum-free media and incubated at 37° C. for 30-60 min. Cells were incubated with ligand for 10 min and then placed on ice and washed with ice-cold PBS. The cells were lysed in 50 mM Tris-HCl (pH 7.4), 150 mM NaCl, 1% Triton X-100, 1 mM EDTA, 1 mM phenylmethylsulfonyl fluoride, 0.5 mM Na₃VO₄, 1 μg/ml leupeptin, 1 μg/ml pepstatin, and 1 μg/ml aprotinin on ice for 10 min. The lysate was clarified by centrifugation at 4° C. Solubilized RON was then immunoprecipitated from the lysate. Antibody RON, clone C-20 (Santa Cruz Biotechnology, Santa Cruz, Calif.) or IMC-41A10 were incubated with 400 μl of lysate at 4 μg/ml overnight at 4° C. Immune complexes were precipitated by the addition of protein A-agarose beads for 2 h at 4° C., pelleted, and washed three times with lysis buffer. Immunoprecipitates bound to the protein A-agarose beads were stripped into denaturing gel sample buffer. Lysates or immunoprecipitates were processed for denaturing gel electrophoresis and run on a 4-12% acrylamide gel and blotted to nitrocellulose membrane by Western blot. Tyrosine-phosphorylated protein was detected on the blot using an anti-phosphoRON antibody (Biosource) and an anti-mouse-horseradish peroxidase secondary antibody. RON was detected with monoclonal antibody RON C-20 (Santa Cruz Biotechnology. Phospho-Akt and total Akt antibodies were obtained from PharMingen (BD Biosciences, San Diego, Calif.). For MAPK phosphorylation, phospho-p44/42 and total p44/42 antibodies were purchased from Cell Signaling Technology). Bands were visualized with the enhanced chemiluminescence reagent (Amersham Pharmacia Biotech) on X-ray film (Eastman Kodak, Rochester, N.Y.).

ELISA for Determination of IC50 and ED50 The ability of the anti-RON antibodies, IMC-41A10 and IMC-41B12, to bind to recombinant human RON receptor and to block the MSP/RON interaction were measured using ELISA. With the receptor immobilized to an ELISA plate, the ED50 values for binding of IMC-41A10 and IMC-41B12 to RON were 0.15 nM and 0.10 nM respectively. Using the same ELISA format, an IC50 value of 2 nM was shared by IMC-41A10 and IMC-41B12 for their ability to block the MSP/RON interaction.

Cell Proliferation Assay. For proliferation inhibition, 10,000 cells from cancer cell lines were seeded into 24-well plates in complete medium. After 24 h, 100 nM anti-RON IMC-41A10 antibody was added to plates in triplicate and allowed to culture for an additional 3 days. The total number of cells (bound and suspension) for each well was determined using a Coulter counter.

Human Tumor Xenograft Model. Tumor xenografts were established by s.c. injection of 5×10⁶ HT-29 cells mixed in Matrigel (Collaborative Research Biochemicals, Bedford, Mass.) into the left flank of 5-6-week-old female athymic (nu/nu) mice (Charles River Laboratories, Wilmington, Mass.). Tumors were allowed to reach 150-300 mm³ in size, and then mice were randomized into groups of 12 animals each. Mice were treated by i.p. injection every 3 days with control antibody (human IgG) or monoclonal anti-RON IMC-41A10 antibody at a dose of 1 mg. Treatment of animals was continued for the duration of the study. Tumors were measured twice each week with calipers, and tumor volumes were calculated by the following formula: (π/6 (w1×w2×w2)), where w1 represents the largest tumor diameter, and w2 represents the smallest tumor diameter. Tumor volumes were analyzed using the Mann-Whitney U test and computed using the statistical package in SigmaStat (version 2.03; Jandel Scientific, San Rafeal, Calif.).
1) Wang, M. H., Kurtz, A. L., Chen, Y. (2000b). Identification of a novel splicing product of the RON receptor tyrosine kinase in human colorectal carcinoma cells. Carcinogenesis 21, 1507-1512.
2) Leonard, E. J., Danilkovitch, A. (2000). Macrophage stimulating protein. Adv. Cancer Res. 77, 139-167.
3) Skeel, A., Leonard, E. J. (1994). Action and target cell specificity of human macrophage-stimulating protein (MSP). J. Immunol. 152, 4618-4623.
4) Leonard, E. J., Skeel, A. (1976). A serum protein that stimulates macrophage movement, chemotaxis and spreading. Exp. Cell Res. 102, 434-438.
5) Iwama, A., Wang, M. H., Yamaguchi, N., Ohno, N., Okano, K., Sudo, T., Takeya, M., Gervais, F., Morissette, C., Leonard, E. J. (1995). Terminal differentiation of murine resident peritoneal macrophages is characterized by expression of the STK protein tyrosine kinase, a receptor for macrophage-stimulating protein. Blood 86, 3394-3403.
6) Wang, M. H., Cox, G. W., Yoshimura, T., Sheffler, L. A., Skeel, A., Leonard, E. J. (1994a). Macrophage-stimulating protein inhibits induction of nitric oxide production by endotoxin- or cytokine-stimulated mouse macrophages. J. Biol. Chem. 269, 14027-14031.
7) Wang, M. H., Dlugosz, A. A., Sun, Y., Suda, T., Skeel, A., Leonard, E. J. (1996a). Macrophage-stimulating protein induces proliferation and migration of murine keratinocytes. Exp. Cell Res. 226, 39-46.
8) Wang, M. H., Montero-Julian, F. A., Dauny, I., Leonard, E. J. (1996b). Requirement of phosphatidylinositol-3 kinase for epithelial cell migration activated by human macrophage stimulating protein. Oncogene 13, 2167-2175.
9) Okino, T., Egami, H., Ohmachi, H., Takai, E., Tamori, Y., Nakagawa, K., Nakano, S., Akagi, J., Sakamoto, O., Suda, T., Ogawa, M. (1999). Presence of RON receptor tyrosine kinase and its splicing variant in malignant and non-malignant human colonic mucosa. Int. J. Oncol. 15, 709-714.
10) Chen, Y. Q., Zhou, Y. Q., Angeloni, D., Kurtz, A. L., Qiang, X. Z., Wang, M. H. (2000). Overexpression and activation of the RON receptor tyrosine kinase in a panel of human colorectal carcinoma cell lines. Exp. Cell Res. 261, 229-238.
11) Wang, M. H., Kurtz, A. L., Chen, Y. (2000b). Identification of a novel splicing product of the RON receptor tyrosine kinase in human colorectal carcinoma cells. Carcinogenesis 21, 1507-1512.
12) Willett, C. G., Wang, M. H., Emanuel, R. L., Graham, S. A., Smith, D. I., Shridhar, V., Sugarbaker, D. J., Sunday, M. E. (1998). Macrophage-stimulating protein and its receptor in non-small-cell lung tumors: induction of receptor tyrosine phosphorylation and cell migration. Am. J. Respir. Cell Mol. Biol. 18, 489-496.
13) Maggiora, P., Marchio, S., Stella, M. C., Giai, M., Belfiore, A., De Bortoli, M., Di Renzo, M. F., Costantino, A., Sismondi, P., Comoglio, P. M. (1998). Overexpression of the RON gene in human breast carcinoma. Oncogene 16, 2927-2933.
14) Chen, Y Q, Zhou, Y Q, Fisher, J H, Wang M-H. (2002). Targeted expression of the receptor tyrosine kinase RON in distal lung epithelial cells results in multiple tumor formation: oncogenic potential of RON in vivo. Oncogene 21, 6382-6386.
15) Chen Y Q, Zhou Y Q, Fu L H, Wang D, Wang M H. (2002). Multiple pulmonary adenomas in the lung of transgenic mice overexpressing the RON receptor tyrosine kinase. Carcinogenesis 23, 1811-1819.
16) Santoro M M, Collesi C, Grisendi S, Gaudino G, Comoglio P (1996). Constitutive Activation of the RON Gene Promotes Invasive Growth but Not Transformation. Molecular and Cellular Biology, December 1996, p. 7072-7083.

1. A monoclonal antibody, or fragment thereof, specific for RON comprising one or more heavy chain CDR sequences selected from the group consisting of SEQ ID NO: 2 (SYAMH) for CDR1; SEQ ID NO: 4 (VISYDGSNKYYADSVKG) for CDR2 and SEQ ID NO: 6 for CDR3 (FSGWPNNYYYYGMDV).

2. The monoclonal antibody, or fragment thereof, of claim 1, wherein the antibody comprises the CDR1, CDR2 and CDR3 sequences.

3. A monoclonal antibody, or a fragment thereof, specific for RON comprising one or more light chain CDR sequences selected from the group consisting of SEQ ID NO: 11 for CDR1 (RSSQSLLHSNGFNYVD); SEQ ID NO: 13 for CDR2 (FGSYRAS) and SEQ ID NO: 15 for CDR3 (MQALQTPPWT).

4. The monoclonal antibody, or fragment thereof, of claim 3, wherein the antibody comprises the CDR1, CDR2 and CDR3 sequences.

5. The monoclonal antibody, or fragment thereof, of claim 4, wherein the antibody comprises a heavy chain variable region sequence of SEQ ID NO:7 or a light chain variable region sequence of SEQ ID NO:16.

6. The monoclonal antibody, or fragment thereof, of claim 5, wherein the antibody comprises both the heavy and light chain with said sequences.

7. The monoclonal antibody, or fragment thereof, of claim 6, wherein the antibody comprises a heavy chain sequence of SEQ ID NO:9 and a light chain sequence of SEQ ID. NO:18.

8. A monoclonal antibody, or a fragment thereof, specific for RON comprising one or more light chain CDR sequences selected from the group consisting of SEQ ID NO: 50 for CDR1 (RSSQSLLHSNGYNYLD); SEQ ID NO: 52 for CDR2 (LGSNRAS) and SEQ ID NO: 54 for CDR3 (MQALQTPRT).

9. The monoclonal antibody, or fragment thereof, of claim 8, wherein the antibody comprises the CDR1, CDR2 and CDR3 sequences.

10. The monoclonal antibody, or fragment thereof, of claim 9, wherein the antibody comprises a heavy chain variable region sequence of SEQ ID NO:41 or a light chain variable region sequence of SEQ ID NO:42.

11. The monoclonal antibody, or fragment thereof, of claim 10, wherein the antibody comprises both the heavy and light chain with said sequences.

12. The monoclonal antibody, or fragment thereof, of claim 11, wherein the antibody comprises a heavy chain sequence of SEQ ID NO:56 and a light chain sequence of SEQ ID NO:58.

13. A monoclonal antibody, or fragment thereof, specific for RON comprising one or more heavy chain CDR sequences selected from the group consisting of SEQ ID NO: 20 (SHYWS) for CDR1; SEQ ID NO: 22 (YIYYSGSTNYNPSLKS) for CDR2 and SEQ ID NO: 24 for CDR3 (IPNYYDRSGYYPGYWYFDL).

14. The monoclonal antibody, or fragment thereof, of claim 13, wherein the antibody comprises the CDR1, CDR2 and CDR3 sequences.

15. A monoclonal antibody, or fragment thereof, specific for RON comprising one or more light chain CDR sequences selected from the group consisting of SEQ ID NO: 29 for CDR1 (TLRSGFNVDSYRIS); SEQ ID NO: 32 for CDR2 (YKSDSDK) and SEQ ID NO: 34 for CDR3 (MIWHSSAWV).

16. The monoclonal antibody, or fragment thereof, of claim 15, wherein the antibody comprises the CDR1, CDR2 and CDR3 sequences.

17. The monoclonal antibody, or fragment thereof, of claim 16, wherein the antibody comprises a heavy chain variable region sequence of SEQ ID NO:25 or a light chain variable region sequence of SEQ ID NO:35.

18. The monoclonal antibody, or fragment thereof, of claim 17, wherein the antibody comprises both the heavy and light chain variable regions with said sequences.

19. The monoclonal antibody, or fragment thereof, of claim 18, wherein the antibody has a heavy chain sequence of SEQ ID NO: 27

20. The monoclonal antibody, or fragment thereof, of claim 19, wherein the antibody has a light chain sequence of SEQ ID NO: 37 or 39.

21. An isolated nucleic acid molecule comprising the nucleic acid sequence selected from the group consisting of SEQ ID NO: 1, 3, 5, 8, 10, 12, 14, 17, 19, 21, 23, 26, 29, 31, 33, 36, 38, 43, 45, 47, 49, 51, 53, 55 and 57.

22. An expression vector comprising the nucleic acid of claim 21 operably linked to a control sequence.

23. A host cell comprising the expression vector of claim 22.

24. A method for producing an antibody comprising culturing the host cell of claim 23 under conditions permitting expression of the antibody.

25. A pharmaceutical composition comprising the monoclonal antibody, or fragment thereof, of any one of claims 1 through 20 and a pharmaceutically acceptable carrier.

26. A method for detecting the presence of RON in a sample comprising contacting said sample with the antibody, or a fragment thereof of any of claims 1 to 20 to obtain specific binding, and detecting such binding.

27. A method for inhibiting growth of mammalian tumor cells that express RON, comprising administering to a mammal an effective amount of an antibody or a fragment thereof specific for RON.

28. A method for inhibiting metastatic activity of mammalian tumor cells that express RON, comprising administering to a mammal an effective amount of an antibody or a fragment thereof specific for RON.

29. A method for treating inflammation mediated by RON activity in a mammal comprising administering to the mammal an antibody or an antibody fragment specific for RON.

30. The method for any one of claims 27 to 29, further comprising administering a small organic molecule, wherein the small organic molecule is a chemotherapeutic agent, anti-angiogenesis agent or inhibitor of RON activation.

31. The method of claim 30, wherein the antibody is conjugated to the small organic molecule.

32. The method for any one of claims 27 to 31, further comprising administering one or more antibodies specific to a receptor tyrosine kinase.

33. The method for claim 32, wherein the receptor tyrosine kinase is EGFR or VEGFR.

34. The method for any one of claims 27, 28, 30-33, wherein the tumor cells are selected from the group consisting of colon, pancreatic, prostate, stomach, lung, liver, ovarian, kidney, breast and brain.

35. The method of claim 34, wherein the tumor cell is from the colon.

36. The method of any one of claims 27, 28, 30-33, wherein the tumor cell is an epithelial cell or a neuroendocrine cell.

37. The method of any one of claims 27 to 36, wherein the RON specific antibody or a fragment thereof is a human antibody.

38. The method of any one of claims 27 to 37, wherein the antibody blocks binding of MSP to RON.

39. The method of any one of claims 27 to 38, wherein the antibody is administered at a dose of about 1 to about 10 mg/Kg.

40. The method of claim 39, wherein the antibody is administered at a dose of about 3 to about 8 mg/Kg.

41. The method of any one of claims 27 to 40, wherein the antibody comprises one or more heavy chain CDR sequences selected from the group consisting of SEQ ID NO: 2 (SYAMH) for CDR1; SEQ ID NO: 4 (VISYDGSNKYYADSVKG) for CDR2 and SEQ ID NO: 6 for CDR3 (FSGWPNNYYYYGMDV).

42. The method of any one of claims 41, wherein the antibody comprises the CDR1, CDR2 and CDR3 sequences.

43. The method of any one of claims 27 to 40, wherein the antibody comprises one or more light chain CDR sequences selected from the group consisting of SEQ ID NO: 11 for CDR1 (RSSQSLLHSNGFNYVD); SEQ ID NO: 13 for CDR2 (FGSYRAS) and SEQ ID NO: 15 for CDR3 (MQALQTPPWT).

44. The method of claim 43, wherein the antibody comprises the CDR1, CDR2 and CDR3 sequences.

45. The method of claim 44, wherein the antibody comprises a heavy chain variable region sequence of SEQ ID NO:7 or a light chain variable region sequence of SEQ ID NO:16.

46. The method of any one of claims 27 to 40, wherein the antibody comprises one or more light chain CDR sequences selected from the group consisting of SEQ ID NO: 50 for CDR1 (RSSQSLLHSNGYNYLD); SEQ ID NO: 52 for CDR2 (LGSNRAS) and SEQ ID NO: 54 for CDR3 (MQALQTPRT).

47. The method of claim 46, wherein the antibody comprises the CDR1, CDR2 and CDR3 sequences.

48. The method of claim 47, wherein the antibody comprises a heavy chain variable region sequence of SEQ ID NO:41 or a light chain variable region sequence of SEQ ID NO:42.

49. The method of any one of claims 27 to 40, wherein the antibody comprises one or more heavy chain CDR sequences selected from the group consisting of SEQ ID NO: 20 (SHYWS) for CDR1; SEQ ID NO: 22 (YIYYSGSTNYNPSLKS) for CDR2 and SEQ ID NO: 24 for CDR3 (IPNYYDRSGYYPGYWYFDL).

50. The method of claim 49, wherein the antibody comprises the CDR1, CDR2 and CDR3 sequences.

51. The method of any one of claims 27 to 40, wherein the antibody comprises one or more light chain CDR sequences selected from the group consisting of SEQ ID NO: 29 for CDR1 (TLRSGFNVDSYRIS); SEQ ID NO: 32 for CDR2 (YKSDSDK) and SEQ ID NO: 34 for CDR3 (MIWHSSAWV).

52. The method of claim 51, wherein the antibody comprises the CDR1, CDR2 and CDR3 sequences.

53. The method of claim 52, wherein the antibody comprises a heavy chain variable region sequence of SEQ ID NO:25 or a light chain variable region sequence of SEQ ID NO:35.

54. The method of claim 53, wherein the antibody comprises both the heavy and light chain variable regions with said sequences.

55. The method of any one of claims 54, wherein the antibody has a heavy chain sequence of SEQ ID NO: 27 and a light chain sequence of SEQ ID NO: 37 or 39.

56. A therapeutic composition for inhibition of growth of tumor cells that express RON in a mammal comprising an antibody, or fragment thereof, specific for RON.

57. The therapeutic composition of claim 56, wherein the antibody or a fragment thereof is a human antibody.


SEQUENCE LISTING (71 sequences)

SEQ ID NO: 1 - DNA, 15 bp, Homo sapiens
agctatgctatgcac

SEQ ID NO: 2 - protein, 5 aa, Homo sapiens
SYAMH

SEQ ID NO: 3 - DNA, 51 bp, Homo sapiens
gttatatcatatgatggaagtaataaatactacgcagactccgtgaagggc

SEQ ID NO: 4 - protein, 16 aa, Homo sapiens
VISYDGSNKYYADSVK

SEQ ID NO: 5 - DNA, 45 bp, Homo sapiens
ttcagtggctggcccaacaactactactactacggtatggacgtc

SEQ ID NO: 6 - protein, 15 aa, Homo sapiens
FSGWPNNYYYYGMDV

SEQ ID NO: 7 - protein, 124 aa, Homo sapiens
EVQLVQSGGGLVKPGGSLRLSCAASGFTFSSYAMHWVRQAPGKGLEWVAVISYDGSNKYYADSVKGRFTISRDNSKNTLYLQMNSLRAEDTAVYYCARFSGWPNNYYYYGMDVWGQGTTVTVSS

SEQ ID NO: 8 - DNA, 1422 bp, Homo sapiens
atgggatggtcatgtatcatcctttttctagtagcaactgcaactggagtacattcagaggtccagctggtgcagtctgggggaggcttggtcaagcctggagggtccctgagactctcctgtgcagcctctggattcaccttcagtagctatgctatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgaggttcagtggctggcccaacaactactactactacggtatggacgtctggggccaagggaccacggtcaccgtctcaagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 9 - protein, 454 aa, Homo sapiens
EVQLVQSGGGLVKPGGSLRLSCAASGFTFSSYAMHWVRQAPGKGLEWVAVISYDGSNKYYADSVKGRFTISRDNSKNTLYLQMNSLRAEDTAVYYCARFSGWPNNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 48 bp, Homo sapiens
aggtctagtcagagcctcctgcatagtaatggattcaactatgtggat

SEQ ID NO: 11 - protein, 16 aa, Homo sapiens
RSSQSLLHSNGFNYVD

SEQ ID NO: 12 - DNA, 21 bp, Homo sapiens
ttcggttcttatcgggcctcc

SEQ ID NO: 13 - protein, 7 aa, Homo sapiens
FGSYRAS

SEQ ID NO: 14 - DNA, 30 bp, Homo sapiens
atgcaagctctgcaaactcctccctggacg

SEQ ID NO: 15 - protein, 10 aa, Homo sapiens
MQALQTPPWT

SEQ ID NO: 16 - protein, 113 aa, Homo sapiens
DVVMTQSPLSLPVTPGEPASISCRSSQSLLHSNGFNYVDWYLQKPGQSPHLLIYFGSYRASGVPDRFSGSGSGTDFTLKISRVEAEDVGVYYCMQALQTPPWTFGQGTKVEIR

SEQ ID NO: 17 - DNA, 720 bp, Homo sapiens
atgggatggtcatgtatcatcctttttctagtagcaactgcaactggagtacattcagatgttgtgatgactcagtctccactctccctgcccgtcacccctggagagccggcctccatctcctgcaggtctagtcagagcctcctgcatagtaatggattcaactatgtggattggtacctgcagaagccagggcagtctccacacctcttgatctatttcggttcttatcgggcctccggggtccctgacaggttcagtggcagtggatcaggcacagattttacactgaaaatcagcagagtggaggctgaggatgttggggtttattactgcatgcaagctctgcaaactcctccctggacgttcggccaagggaccaaggtggaaatcagacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 18 - protein, 220 aa, Homo sapiens
DVVMTQSPLSLPVTPGEPASISCRSSQSLLHSNGFNYVDWYLQKPGQSPHLLIYFGSYRASGVPDRFSGSGSGTDFTLKISRVEAEDVGVYYCMQALQTPPWTFGQGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 15 bp, Homo sapiens
agtcactactggagt

SEQ ID NO: 20 - protein, 5 aa, Homo sapiens
SHYWS

SEQ ID NO: 21 - DNA, 48 bp, Homo sapiens
tacatctattacagtgggagcaccaactacaacccctccctcaagagt

SEQ ID NO: 22 - protein, 16 aa, Homo sapiens
YIYYSGSTNYNPSLKS

SEQ ID NO: 23 - DNA, 57 bp, Homo sapiens
attcccaactactatgatagaagtggttattatcccggttactggtacttcgatctc

SEQ ID NO: 24 - protein, 19 aa, Homo sapiens
IPNYYDRSGYYPGYWYFDL

SEQ ID NO: 25 - protein, 127 aa, Homo sapiens
QVQLQESGPGLVKPSEILSLTCTVSGGSISSHYWSWVRQPPGKGLEWIGYIYYSGSTNYNPSLKSRVTISVDTSKNQFSLNLSSVTAADTAVYYCARIPNYYDRSGYYPGYWYFDLWGRGTLVTVSS

SEQ ID NO: 26 - DNA, 1431 bp, Homo sapiens
atgggatggtcatgtatcatcctttttctagtagcaactgcaactggagtacattcacaggtgcagctgcaggagtccggcccaggactggtgaagccttcggagatcctgtccctcacctgcactgtctctggtggctccatcagtagtcactactggagttgggtccggcagcccccagggaagggactggagtggattgggtacatctattacagtgggagcaccaactacaacccctccctcaagagtcgagtcaccatatcagtagacacgtccaagaaccagttctccctgaacctgagctctgtgaccgctgcggacacggccgtgtattattgtgcgagaattcccaactactatgatagaagtggttattatcccggttactggtacttcgatctctggggccgtggcaccctggtcaccgtctcaagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 27 - protein, 457 aa, Homo sapiens
QVQLQESGPGLVKPSEILSLTCTVSGGSISSHYWSWVRQPPGKGLEWIGYIYYSGSTNYNPSLKSRVTISVDTSKNQFSLNLSSVTAADTAVYYCARIPNYYDRSGYYPGYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - skipped

SEQ ID NO: 29 - protein, 14 aa, Homo sapiens
TLRSGFNVDSYRIS

SEQ ID NO: 30 - DNA, 42 bp, Homo sapiens
accttgcgcagtggcttcaatgttgattcctacaggatatcc

SEQ ID NO: 31 - DNA, 21 bp, Homo sapiens
tacaaatcagactcagataag

SEQ ID NO: 32 - protein, 7 aa, Homo sapiens
YKSDSDK

SEQ ID NO: 33 - DNA, 27 bp, Homo sapiens
atgatttggcacagcagcgcttgggtg

SEQ ID NO: 34 - protein, 9 aa, Homo sapiens
MIWHSSAWV

SEQ ID NO: 35 - protein, 117 aa, Homo sapiens
QAVLTQPSSLSAPPGASASLTCTLRSGFNVDSYRISWYQQKPGSPPQYLLRYKSDSDKQQGSGVPSRFSGSKDASANAGILLISGLQSEDEADYYCMIWHSSAWVFGGGTKLTVLRT

SEQ ID NO: 36 - DNA, 726 bp, Homo sapiens
atgggatggtcatgtatcatcctttttctagtagcaactgcaactggagtacattcacaggctgtgctgactcagccgtcttccctctctgcacctcctggagcatcagccagtctcacctgcaccttgcgcagtggcttcaatgttgattcctacaggatatcctggtaccagcagaagccagggagtcctccccagtatctcctgaggtacaaatcagactcagataagcagcagggctctggagtccccagccgcttctctggatccaaagatgcttcggccaatgcagggattttactcatctctgggctccagtctgaggatgaggctgactattactgtatgatttggcacagcagcgcttgggtgttcggcggagggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 37 - protein, 222 aa, Homo sapiens
QAVLTQPSSLSAPPGASASLTCTLRSGFNVDSYRISWYQQKPGSPPQYLLRYKSDSDKQQGSGVPSRFSGSKDASANAGILLISGLQSEDEADYYCMIWHSSAWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 723 bp, Homo sapiens
atgggatggtcatgtatcatcctttttctagtagcaactgcaactggagtacattcacaggctgtgctgactcagccgtcttccctctctgcacctcctggagcatcagccagtctcacctgcaccttgcgcagtggcttcaatgttgattcctacaggatatcctggtaccagcagaagccagggagtcctccccagtatctcctgaggtacaaatcagactcagataagcagcagggctctggagtccccagccgcttctctggatccaaagatgcttcggccaatgcagggattttactcatctctgggctccagtctgaggatgaggctgactattactgtatgatttggcacagcagcgcttgggtgttcggcggagggaccaagctgaccgtcctaagtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctgcagaatgctcttga

SEQ ID NO: 39 - protein, 221 aa, Homo sapiens
QAVLTQPSSLSAPPGASASLTCTLRSGFNVDSYRISWYQQKPGSPPQYLLRYKSDSDKQQGSGVPSRFSGSKDASANAGILLISGLQSEDEADYYCMIWHSSAWVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 40 - protein, 115 aa, Homo sapiens
QAVLTQPSSLSAPPGASASLTCTLRSGFNVDSYRISWYQQKPGSPPQYLLRYKSDSDKQQGSGVPSRFSGSKDASANAGILLISGLQSEDEADYYCMIWHSSAWVFGGGTKLTVL

SEQ ID NO: 41 - protein, 124 aa, Homo sapiens; identical to SEQ ID NO: 7

SEQ ID NO: 42 - protein, 112 aa, Homo sapiens
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIK

SEQ ID NO: 43 - DNA, 15 bp, Homo sapiens
agctatgctatgcac

SEQ ID NO: 44 - protein, 5 aa, Homo sapiens
SYAMH

SEQ ID NO: 45 - DNA, 51 bp, Homo sapiens; identical to SEQ ID NO: 3

SEQ ID NO: 46 - protein, 17 aa, Homo sapiens
VISYDGSNKYYADSVKG

SEQ ID NO: 47 - DNA, 45 bp, Homo sapiens; identical to SEQ ID NO: 5

SEQ ID NO: 48 - protein, 15 aa, Homo sapiens
FSGWPNNYYYYGMDV

SEQ ID NO: 49 - DNA, 48 bp, Homo sapiens
aggtctagtcagagcctcctgcatagtaatggatacaactatttggat

SEQ ID NO: 50 - protein, 16 aa, Homo sapiens
RSSQSLLHSNGYNYLD

SEQ ID NO: 51 - DNA, 21 bp, Homo sapiens
ttgggttctaatcgggcctcc

SEQ ID NO: 52 - protein, 7 aa, Homo sapiens
LGSNRAS

SEQ ID NO: 53 - DNA, 27 bp, Homo sapiens
atgcaagctctacaaactcctcggacg

SEQ ID NO: 54 - protein, 9 aa, Homo sapiens
MQALQTPRT

SEQ ID NO: 55 - DNA, 372 bp, Homo sapiens
gaggtccagctggtgcagtctgggggaggcttggtcaagcctggagggtccctgagactctcctgtgcagcctctggattcaccttcagtagctatgctatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgaggttcagtggctggcccaacaactactactactacggtatggacgtctggggccaagggaccacggtcaccgtctcaagc

SEQ ID NO: 56 - protein, 454 aa, Homo sapiens; identical to SEQ ID NO: 9

SEQ ID NO: 57 - DNA, 336 bp, Homo sapiens
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaa

SEQ ID NO: 58 - protein, 219 aa, Homo sapiens
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 4203 bp, Homo sapiens
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctggcagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcaggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccgaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgagtatattgggctgggcgctgtggctgactgtgtgggtatcaacgtgaccgtgggtggtgagagctgccagcacgagttccggggggacatggttgtctgccccctgcccccatccctgcagcttggccaggatggtgccccattgcaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggccactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 60 - protein, 1400 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAGSFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLQVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIRGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRPVPRKDFVEEFECELEPLGTQAVGPTNVSLTVTNMPPGKHFRVDGTSVLRGFSFMEPVLIAVQPLFGPRAGGTCLTLEGQSLSVGTSRAVLVNGTECLLARVSEGQLLCATPPGATVASVPLSLQVGGAQVPGSWTFQYREDPVVLSISPNCGYINSHITICGQHLTSAWHLVLSFHDGLRAVESRCERQLPEQQLCRLPEYVVRDPQGWVAGNLSARGDGAAGFTLPGFRFLPPPHPPSANLVPLKPEEHAIKFEYIGLGAVADCVGINVTVGGESCQHEFRGDMVVCPLPPSLQLGQDGAPLQVCVDGECHILGRVVRPGPDGVPQSTLLGILLPLLLLVAALATALVFSYWWRRKQLVLPPNLNDLASLDQTAGATPLPILYSGSDYRSGLALPAIDGLDSTTCVHGASFSDSEDESCVPLLRKESIQLRDLDSALLAEVKDVLIPHERVVTHSDRVIGKGHFGVVYHGEYIDQAQNRIQCAIKSLSRITEMQQVEAFLREGLLMRGLNHPNVLALIGIMLPPEGLPHVLLPYMCHGDLLQFIRSPQRNPTVKDLISFGLQVARGMEYLAEQKFVHRDLAARNCMLDESFTVKVADFGLARDILDREYYSVQQHRHARLPVKWMALESLQTYRFTTKSDVWSFGVLLWELLTRGAPPYRHIDPFDLTHFLAQGRRLPQPEYCPDSLYQVMQQCWEADPAVRPTFRVLVGEVEQIVSALLGDHYVQLPATYMNLGPSTSHEMNVRPEQPQFSPMPGNVRRPRPLSEPPRPT

SEQ ID NO: 61 - protein, 5 aa, Homo sapiens
SYAIS

SEQ ID NO: 62 - protein, 17 aa, Homo sapiens
GIIPIFGTANYAQKFQG

SEQ ID NO: 63 - protein, 21 aa, Homo sapiens
APLRFLEWSTQDHYYYYYMDV

SEQ ID NO: 64 - protein, 11 aa, Homo sapiens
QGDSLRSYYAS

SEQ ID NO: 65 - protein, 7 aa, Homo sapiens
GKNNRPS

SEQ ID NO: 66 - protein, 11 aa, Homo sapiens
NSRDNSDNRLI

SEQ ID NO: 67 - protein, 11 aa, Homo sapiens
QGDSLRSYYAT

SEQ ID NO: 68 - protein, 7 aa, Homo sapiens
GENKRPS

SEQ ID NO: 69 - protein, 11 aa, Homo sapiens
KSRDGSGQHLV

SEQ ID NO: 70 - DNA, 23 bp, Artificial Sequence
  Description of Artificial Sequence: Synthetic primer
agcggataacaatttcacacagg

SEQ ID NO: 71 - DNA, 21 bp, Artificial Sequence
  Description of Artificial Sequence: Synthetic primer
gtcgtctttccagacgttagt